SEQUENCE LISTING (6 sequences)

SEQ ID NO: 1 - DNA, 607 bp, Homo sapiens
ttgaatttaggtgacactatagaagagctatgacgtcgcatgcacgcgtacgtaagcttggatcctctagagcggccgcctactactactaaaattcgcggccgcgtcgacaggaaattaatgaggattacttttgagagtctgacatgcatatcataattttatgtcaggtattatagatattttgaaatggtgactgactcttttgaaattttaagttctttagaatgtgacgcttttaatatagcctctggttttagatggagaaacactatgctattgtcattaaaaattaattctatttccccaattgtctaatatatgtcttaaaagatctttcatattgtgaaacatcagagggtacaacctttgttcttcagtttaggtattaaagagcacacagaatactgtgtgattaaacatgtaaggccagataatgcatttgcaaaggttcctttattttaggtttaagcctgcataattgtggtcttaatctcaggatagcaagaaagagaattgtacatgaaagtatttacacaaagttcccaaagcccttgtgattatgcattagtttagaataaaaaaaaaaaaaaagtcgacgccgccg

SEQ ID NO: 2 - DNA, 371 bp, Homo sapiens
tttatgttccaatacaactttatttgcaaaaacaggtggatttggcctccagagccagagttttccaacccctgagataaagcaaacattgctttcataaatgttcacaattgtaaaaatataaagttcaattaaattttacatttacttagatttattagccagaatttttataaaaggattgatttttataacttcatataatgctgcaattatgtgactctatagattttaccttatttaaaaattttaaatgtctatcaattttaaaaatcaacagtaattgtattgctatttaaggatcactttaaattataagatctccaattcacttagacaaaatgatcttgagagttactaataaataattt

SEQ ID NO: 3 - DNA, 2186 bp, Homo sapiens
tttctccgggggcgggctgcaataaaacagcaaaatcctccattccccatcagcggtcgattgggggctctttcgaagcggcagctctgtaggaccgctcttctaccccaagatccctcggcttcggccccctaggtgccagggtctgcgggaccccacgctcaaagccgccggccggggtcgacaactgcagagagggtcgggataggaaattgcgggtagcggcagatgcgggtccccaggcatcccggaggttcccacaggtctgcagtgggcctactttgcgaaaagggcccggctgcgccgagcctcgttcaaatcagatgccagacagtgtttcttgggatccatccaaataggtcccttatttctctttgtgggagccctgggcctccttccaggccggaacccaagtgcttaggcagccgggaaaggccggtcccctttttcagttctctcgcgacctctagccacttccggttgctaacggttcccaaacagcccccgaaaacgctacgtgagctgggccctgggccagaggcagaaaacggacggaagaaaaggtctggccggttcatcaagctctctctccagatcctccagtaccgtcactgcctcctctccagtctctggatggtgtgcacgcacccatgcttgcaggtctttaggaagaatgctcaggaactgttctagcaccagcaggtctaaaatctgctccttggtgtggcattctggcctcagccactgacggcagagctcccacagctgggagatgggtctcactctgtcacccagactggagtgcagtgagtggtgcgatcatagcttactgcagcctgaaactcctgggctcaagtgatcttctcgcctcagcctcctgagtagctggagctacaggtgtgagctacccagcatggctcatttgagatttctgagtagagaagtaacatgattaaacttgggtattgagattattattttggctgctatgttaacagtagacttgaatgtgaaggggttgggcaagggcagaagcagggagacaggttggaaagttggagtgggaagggcctttttaagaataacacaaacccctaaagacataaaactgaaaaggccatggaggaaaagataaatgaaactggccttgtaaaattgaaatatttgaatgaaaaaagtaaaaataaaatataaactaaaagtaaaatatgccacacatctaaaagacacagctgattttcctgatatacaaagaccttttatcttaccaaaaaataaaaatatgaacaagccaatagaaaaatagacaaagtacttaacctgcatgcatttattgtcaggcctctgacaatatgatactaagccatcatagcccctgtgaccgccacgtatacatccagatgacctggagcaactgaagaaccacaaaagatggcattccaccattgagatttgttcctgccccaccccaactaatcaatcgaccttgtgacattccccctgcccccgacagtgagtctcatgatctccccacccagcaccttggcaccttgtgacccccgcccctgcccacaagagataaccacctttaactgtaattttccactacctacccaaatcctataaaactgccccacccctatctccctttgctgactctctttttggactcagcccacttgcacccaagtgaaataaacagccttgttgctcacacaaagcctgttggtggactctcttcacacagactcacttgacatttatagaacacaaaattaagaatagtgaatatgaaaaggttcactaataagaatttaaacaaaacaagaaagatttttaagattgctaaagacaaacgatttataatattcagtgttaggaggtgtgtggatatacaggcatacttagttttacatttttagaaggtaagacttatttagaaggtaagaagttgtccataccccttgattcaacaacttagcttcttggaattatccatatagaaatgcttacataaatgtttgaacgtatgtacaaagaagcactatttatttaaattgggaaacttgaatgcctaacatttggaatttaaatttttatatgtctattcattaaaataccacccagtttttagaaactaagagatgctaaaggtatattgagtaaaataaaaagttacaaaagat

SEQ ID NO: 4 - DNA, 649 bp, Homo sapiens
gaaagcggtcgtggccaggactggcgcagctgccgtttcaggcgaggggcgcagaatgcagcggccgccagcctggagcgcgggccctggggccgcaacccgcgccgggcgaggtggcagcacaccctgggccccccactccccgccgcaagtcctgaggatggccagcagagaaacaagaaaatggactccctggctgctggagagttgaatgccagccaccagccatgggtgccagagtttgtagcctattggaggaaaacacaccaagatcacctctgcagcctgcacagccgggcctttggactcctggatgctagagtgacctgggcgctgaggagggcccccgagccagtaccaggaaaggatagactcctgcttgcagcattcccagcagaggcatcgcctgtggacaccgcgtctgtgtctgtatatggcagagctcccagatatatgcacaagggagtgaaaaaatgtgtttgcaccccagtctctaaaaattcaacagcctggttacttctgggtggtatatcgtaggtggctttaatacgtgttatttgctcatctgtatttcttactctttgcacaattaaaccatgttccttttacttatgtacatttttaataaaagaaagttgttaatgactca

SEQ ID NO: 5 - DNA, 537 bp, Homo sapiens
tttttttttttttttttagtcaaacaaaatagataacatccaactgcacctgccactgttaaggacagttactgtattatgaaacttcttgttttacccacacacaaacattcatacatgcatatattagatagaagtgtgacatgtatttcttactataggtcatagtttaaaaattaaaacctaactcaccattccccacatctcctgggcttctcacctccaagcctttgcttacgctaatctctcaggcaggaatgctctctttcagtaatctttgccaatcaaaatcctatcaatccaaaacttaaaaaaaaaaatcttatcaatcccttcaaagactagctaaaattccacttttatgcctttaaagttctgtctcaggttttctcctaatcaaatgcattttctgccttttaaattcagtaatatttggtatatttgtgactcacagatatatttgtgattatagtacaggtatttgtgcacaagtccagctgccctaccacaatataaatcccaaaggttttattcctg

SEQ ID NO: 6 - DNA, 568 bp, Homo sapiens
  misc_feature (528)..(528): n is a, c, g, or t
tttaagtatagatggtaatttatttatttaaagatgtaaaaactgtttcaaacaatttgaacctttaaacagaccaggttttagcactcaactgaaatccaatactttctccattcattggcctgatctaatcagacacagtgtagtagtttcctattgctgctgtaacaaattgccacaaacttagtggcctcaaacaacacacattgattatcctgccgttctaagatgggtcagcaaggccgtgctccttctgaaggctccaggaaagaacccatggacttgccttttccagcttctagaggccacctgcatcccactgccgcctgcttcccttggcacagctctttcgtctgcctcagaccttcctgcctccctcttgaaaggatctttgtggttacattaaactcacctgggtaatccaggaatccctccccatcacaaggttcttaatcacagctgccgaatccccttttgccacggaaggtaacatattctcagttccggggattaggatgtgggcctctntgtgggccactattctgtataccccagatagtgattcctg

(19) United States
(12) Patent Application Publication
(10) Pub. No.: US 2022/0002807 A1
(43) Pub. Date: Jan. 6, 2022

(54) SUPERVISED LEARNING METHODS FOR THE PREDICTION OF TUMOR RADIOSENSITIVITY TO PREOPERATIVE RADIOCHEMOTHERAPY

(71) Applicants: H. LEE MOFFITT CANCER CENTER AND RESEARCH INSTITUTE, INC., Tampa, FL (US); UNIVERSITY OF SOUTH FLORIDA, Tampa, FL (US)

(72) Inventors: Florentino A. Rico, Tampa, FL (US); Grisselle Centeno, Lakeland, FL (US); Ludwig Kuznia, Lakeland, FL (US); Steven A. Eschrich, Lakeland, FL (US); Javier F. Torres-Roca, St. Petersberg, FL (US)

(21) Appl. No.: 17/342,106

(22) Filed: Jun. 8, 2021

Related U.S. Application Data
(63) Continuation-in-part of application No. 16/513,230, filed on Jul. 16, 2019, now abandoned.
Application No. 16/513,230 is a continuation of application No. 15/509,044, filed on Mar. 6, 2017, now abandoned.
(60) Provisional application No. 62/049,431, filed on Sep. 12, 2014.
(60) Provisional application No. 62/085,922, filed on Dec. 1, 2014.

(51) Int. Cl. C12Q 1/6883; C12Q 1/6886

(52) U.S. Cl. CPC C12Q 1/6883; C12Q 1/6886; C12Q 2600/112; C12Q 2600/158; C12Q 2600/106

(57) ABSTRACT

Disclosed is a gene expression panel that can predict radiation sensitivity (radiosensitivity) of a tumor in a subject. A method of predicting radiation sensitivity is provided that is based on cellular clonogenic survival after 2 Gy (SF2) for 48 cell lines. Gene expression is used as the basis of the prediction model. The radiosensitivity cell-based prediction model is validated using clinical patient data from rectal and esophagus cancer patients that received RT before surgery. The radiosensitivity genomic-based prediction model identifies patients with rectal cancer that may benefit from RT treatment by assigning higher values of SF2 to radio-resistant patients and lower values of SF2 to radio-sensitive patients.

### CROSS-REFERENCE TO RELATED APPLICATIONS

This application is a continuation-in-part of U.S. application Ser. No. 16/513,230, filed on Jul. 16, 2019, which is a continuation of Ser. No. 15/509,044, filed on Mar. 6, 2017, which is a 35 USC § 371 national stage filing of International PCT Application No. PCT/US2015/049665, filed on Sep. 11, 2015, which claims the benefit of U.S. Provisional Patent Application No. 62/049,431, filed Sep. 12, 2014 and U.S. Provisional Patent Application No. 62/085,922, filed Dec. 1, 2014, each entitled “Supervised Learning Methods for the Prediction of Tumor Radiosensivity to Preoperative Radiochemotherapy.” The disclosures of the aforementioned U.S. Patent Applications are incorporated by reference in their entireties.

### BACKGROUND

Rectal cancer is a disease in which malignant cells form in the tissues of the rectum. As shown in FIG. 1, the rectum is part of the colon and is located in the gastrointestinal track; thus, its position in the pelvis poses additional challenges in treatment when compared with colon cancer. Colorectal cancer is the third most common cancer diagnosed in both men and women in the United States. According to the American Cancer Society, 96,830 new cases of colon cancer and 40,000 new cases of rectal cancer were reported in 2014. However, rates have been declining by 3.0% per year in men and by 2.3% per year in women since 1998. This trend has been attributed to the detection and removal of precancerous polyps as a result of colorectal cancer screening. Overall, only 39% of colorectal cancer patients diagnosed between 1999 and 2006 had localized-stage disease, for which the 5-year relative survival rate is 90%; 5-year survival rates for patients diagnosed at the regional and distant stage are 70% and 12%, respectively. The 5-year observed survival rate for colon and rectal cancer patients between 1998 and 2000 are shown in Table 1 by cancer staged from the 7th edition of the AJCC staging system (from National Cancer Institute's SEER database). The observed estimates in Table 1 may be lower than actual survival rates since it includes patients who could have died from other causes than cancer during the observed timeframe (e.g. heart disease).

TABLE 1
Survival rates for rectal and colon cancer by stage
5-year Observed Survival Rate

| Stage | Colon Cancer (%) | Rectal Cancer (%) |
|---|---|---|
| II | 74 | 74 |
| IIA | 67 | 65 |
| IIB | 59 | 52 |
| IIC | 37 | 32 |
| IIIA | 73 | 74 |
| IIIB | 46 | 45 |
| IIIC | 28 | 33 |
| IV | 6 | 6 |

FIG. 2 illustrates a general process 200 for rectal cancer detection and treatment of colorectal cancer. The process consists of first detecting and diagnosing the cancer (202), determining the stage of the cancer (204), and finally selecting the treatment at 206 (e.g., two or more types of treatment may be combined or used in sequence, as shown by various combinations 208a-208b, 210a-210b, 212, 214 and 216) that is based on the cancer stage prognosis and physician expertise. After treatment, follow up and monitoring is recommended to assess treatment effectiveness and as a preventive measure. In practice, there are algorithms in place that suggests the treatment combination based on the cancer stage and cancer type. An example of treatment selection algorithm for rectal cancer patients is one created by the MD Anderson Cancer Center. Other example treatment selections would be known by one of ordinary skill in the art. Below, process component shown in FIG. 2 is described in detail

At 200, Rectal Cancer Diagnosis is performed. Most people in early colon or rectal cancer stages do not experience the symptoms of the disease. Thus, screening tests are recommended to detect and diagnose the cancer before it further progresses. One or more of tests used to detect and diagnose colon and rectal cancer include:
Endoscopic tests are nonsurgical procedures to examine and remove suspicious tissue or polyps. Depending on how far up the colon is examined, three tests are performed:
   Proctoscopy: to view the rectum
   Sigmoidoscopy: to view of the rectum and lower colon
   Colonoscopy: to view the entire colon
Endoscopic ultrasound: a picture (sonogram) is obtained by bouncing high-energy sound waves (ultrasound) off internal organs
Imaging tests infuse energy through a patient and can show abnormal body structures. Changes in energy patterns are captured to create an image or picture that is reviewed by a physician and include:
   Computed tomography scan (CT)
   Magnetic resonance imaging scan (MRI)
   Positron emission tomography scan (PET)
Digital rectal exam
Carcinoembryonic antigen (CEA) measures the quantity of this protein in the blood of patients who have may have colon or rectal cancer
Fecal occult blood and immunochemical tests

At 204, staging is performed. Staging is the process of determining the spread and extent of the cancer tumor once it has been diagnosed. It is based on the results of the physical exam, biopsies, blood and imaging tests. The American Joint Committee on Cancer (AJCC) staging system, also known as the TNM system, is the tool most commonly staging used for colorectal cancer. The TNM consists of three key elements:
T: defines how much the tumor has grown into the wall of the intestine
N: defines the extent of spread to other lymph nodes
M: defines whether the cancer has metastasized to other organs of the body

Once the patient's T, N and M categories have been determined, a stage grouping (from stage I to stage IV in Error! Reference source not found.) is determined from the least advanced to the most advanced stage.

At 205, treatment options are determined. There are different types of treatment for rectal cancer, some are standard practice and others are being tested in clinical trials. According to the National Cancer Institute (NCI), four types of standard treatment are used: surgery, radiation therapy (RT), chemotherapy, and targeted therapy. There treatments can be performed separately or combined as shown in FIG. 2 at 208a-208b, 210a-210b, 212, 214 and 216. An oncologist will select the best therapy based on the type of cancer, stage and location of the tumor.

The primary treatment used in rectal cancer is surgical resection. According to the NCI, local excision of clinical tumors is commonly used for selected patients in rectal cancer stage T1. For higher stages of rectal cancer, a total mesorectal excision (TME) is the treatment of choice. Since the introduction of TME for rectal cancer, reduced local recurrence rates and improved oncologic outcomes have been observed. Depending on the surgeon's experience, the rate of complications, such as blood loss and anastomotic leaks, are low. Furthermore, radiotherapy before surgery appears to benefit patient outcomes even with improvements in surgical technique.

Radiation Therapy (RT) is the most commonly prescribed treatment in rectal cancer treatment. Approximately 50% of cancer patients will receive RT alone or in combination with other treatments. When used before surgery, the goal is to shrink the tumor to make surgery or chemotherapy more effective. When used afterward, it is used to destroy any cancer cells that might remain after surgery. There are two basic types of RT:
External beam radiation is administered by a machine and rotates around the patient's body to deliver a high dose of radiation directly to the tumor (some of the tissue around the tumor can also be affected).
Internal radiation, also known as brachytherapy, consists of a radiation source that is implanted in the body at the tumor site. Based on the type of the tumor, the appropriate equipment is selected for treatment.

A combination of radiation and chemotherapy before radiation (also known preoperative chemo-radiation (CRT) or neoadjuvant therapy) has become the standard of care for patients with clinically staged T3-T4 or node-positive disease based on the results of clinical trials. CRT may be given before surgery to shrink the tumor, make it easier to remove the cancer, and lessen problems with bowel control after surgery. Even if all the cancer that can be seen at the time of the surgery is removed, some patients may be given radiation therapy or chemotherapy after surgery to kill any cancer cells that are left. Treatment given after the surgery to lower the risk that the cancer will come back is called adjuvant therapy.

For patients with rectal cancer stage II and III, neoadjuvant treatment with RT and 5-FU-based chemotherapy is preferred compared to adjuvant therapy in reducing local recurrence and minimizing toxicity. However, there are specific challenges and adverse effects associated with the RT in rectal cancer patients. These include:
Gastrointestinal disorders: diarrhea, bleeding, abdominal pain and obstruction due to stenosis or adhesions
Genitourinary dysfunction: incontinence, retention, dysuria, frequency and urgency
Sexual Dysfunction: in males, a long-term deterioration of ejaculatory and erectile function; and in females, RT was associated with vaginal dryness and diminished sexual satisfaction
Second Cancers: risk of second cancers from organs within or adjacent to the irradiated target. The most common second cancers include gynecologic and prostate.

RT after or before surgery treatment has negative effects on toxicity and the quality of life of the patient; therefore, treatment options should be discussed with the patient.

Personalized medicine refers to the use and implementation of the patient's unique biologic, clinical, genetic and environmental information to make decisions about their treatment or course of action. Cancer Therapy is implemented on a watch-and-wait basis for most patients. Although an individual's clinical information (cancer stage) is used to decide which regimen is likely to work best, only data referring to outcomes of larger groups of patients is considered herein.

Under the umbrella of personalized medicine is genomic medicine, which refers to “the use of information from genomes (from humans and other organisms) and their derivatives (RNA, proteins, and metabolites) to guide medical decision making,” as described by G. S. Ginsburg and H. F. Willard, “Genomic and personalized medicine: foundations and applications,” Transl. Res., vol. 154, no. 6, pp. 277-87, December 2009. The discovery of patterns in gene expression data and examining a person's genome makes possible to make individualized risk predictions and treatment decisions. A patient predisposition to treatment and health states can now be characterized by their molecular information, and useful classifiers and prognostic models can be developed to more strategically make decisions.

There has been a significant improvement in sensitivity as DNA microarray technology continues to advance. DNA microarray and gene expression profiles data has made possible to understand and make new discoveries at the molecular level regarding human conditions and diseases, especially cancer. However, a challenge facing this area of study is the complexity and amount data across multiple samples.

This research is motivated by the question whether it is possible to determine which patients will more likely benefit from using RT as part of their cancer treatment. Clinical decision-making regarding RT is still based on estimated overall level of tumor aggressiveness, but current decision models are not personalized for predicting the benefit from RT for a specific patient, as described by J. F. Torres-Roca and C. W. Stevens, “Predicting response to clinical radiotherapy: past, present, and future directions,” Cancer Control, vol. 15, no. 2, pp. 151-6, April 2008 (herein “Torres-Roca”). Torres-Roca developed and validated a system biology model of cellular radiosensitivity would lead to the discovery of novel radiation specific predictive biomarkers. The clinical applications of this type of personalized predictive model have the potential to identify patients likely to benefit from certain treatment and determine a more effective treatment strategy.

There has been an increasing trend in the way patients are moving from being a passive actor of their disease management process to actively making decisions regarding their treatment. It could now be expected that patients will at least give true informed consent to their treatment, if not actually making such treatment decisions themselves. Depending in the stage of the cancer, the decision of receiving a treatment is a matter of several factors and implications that influence the patient to accept or reject treatment. Further treatment may prolong life or relieve symptoms, but in some cases will not eradicate the disease. A trade off must be made between possible benefits and likely side effects.

The decision making process should consider the individual patients preferences for which treatment, if any, should be selected. Different significant predictors for overall survival, quality of life, cost-effectiveness, and response to treatment include individual patient genomic profile factors, prognostic biomarkers, and socio-economical patient characteristics. This information can help the patient make a decision, based on their individual preferences and personal situation.

As patients continue to gain control over their treatment strategies, more support is needed to help them make good decisions. It is still unclear to what extend patients are involved in their decision making and how they can resolve their personal uncertainty regarding their treatment options. D. J. Kiesler and S. M. Auerbach, “Optimal matches of patient preferences for information, decision-making and interpersonal behavior: evidence, models and interventions.,” Patient Educ. Couns., vol. 61, no. 3, pp. 319-41, June 2006, reviewed studies regarding the involvement of patients in the decision making process, they found that although a large proportion of patient want to be fully informed and actively participate in their treatment decisions with their physicians, a considerable proportion of patients prefer to have little to no detailed information about their condition or involvement in medical decisions. This shared decision process is dynamic in the sense that it will vary depending on the patient preferences.

Other literature exists that concentrates on decision models used to select which treatment should be selection for patients with cancers. A large of proportion of articles are focused in determining which prognostic factors and biomarkers are the most significant predictors in the assessment of different outputs (e.g. Survival, Recurrence rate and chances of metastasis). The information, criteria, methods and objectives used in the models to make the treatment selection decision are listed in Table 2.

The objectives and criteria used in cancer treatment selection models involve intrinsic trade-offs between survival and quality of life. Summers (2007) assessed trade-offs between quantity and quality of life particular to prostate cancer patients as well as among different side effects to determine which treatment would be optimal for a specific patient [20]. [21], [22], [23], [24], used an utility score and defined it as the relative value patients assign to potential health states. Utilities values were obtained from interviews or the literature. Some of the treatment complications considered include: sexual dysfunction, urinary symptoms bowel dysfunction, and death. Szumacher, 2005 [25], implemented a decision model mainly based on patients preferences in regards to convenience of treatment plan, pain relief, overall quality of life, Individual's chances of survival and out-of-pocket costs. Survival, chance of metastasis and risk of relapse are usually compared to quality of life measures: [26], [27] evaluated models based on the probability of the cancer relapsing after an amount of time, and [20], [24], [27] assessed the chance of the cancer spreading to other organs as decision criteria. On the other hand, A number of articles concentrated specifically on the cost effectiveness of various strategies [28], [29], [27]. Van Gerven, 2007 [30], focused on the maximization of patient benefit, while simultaneously minimizing the cost of treatment.

Among the methods utilized in the literature, different types of Markov decision analysis framework were the most used [29], [21], [20], [22], [30], [23]. A Markov decision process extends a Markov chain by allowing actions and rewards to incorporate both choice and motivation, also the Markov property ensures that the future state is independent of the past state given the current state of a random process. [28], [29], [27] used decision tress and cost-effectiveness analysis as a strategy to select strategies. Multi-criteria optimization models were used in [31], [32] to find the best dose-volume histogram (DVH) values by varying the dose-volume constraints on each of the organs at risk (OARs). Other methods used include: neural networks [25] and multivariate statistical analysis [25]. In most cases, Individual patient risks and preferences are not considered in these models to make individual recommendations. Therefore, future analyses need provide outcomes stratified by more specific risks and preferences.

The Data used as inputs considered in the models include tumor anatomy factors, patients' characteristics, and cost estimates. Tumor anatomy is also considered using the TNM staging system in various studies [30], [28], [24], [29]. Gleason score and prostate-specific antigen (PSA) are important input for prostate cancer treatment selection [21], [20], [22], [24]. Age is the most commonly patients characteristics considered in the models [21], [20], [22], [24], [30], [23], [28], [26], [25]. Other patient and health factors include: gender, race, treatment history, comorbidities, and laboratory results.

Below is a key to the references noted in Table 2 and discussed above:
[20] B. D. Sommers, C. J. Beard, A. V D'Amico, D. Dahl, I. Kaplan, J. P. Richie, and R. J. Zeckhauser, “Decision analysis using individual patient preferences to determine optimal treatment for localized prostate cancer.,” Cancer, vol. 110, no. 10, pp. 2210-7, November 2007.
[21] M. W. Kattan, M. E. Cowen, and B. J. Miles, “A Decision Analysis for Treatment of Clinically Localized Prostate Cancer,” J. Gen. Intern. Med., vol. 12, no. 5, pp. 299-305, 1997.
[22] V. Bhatnagar, S. Stewart, W. Bonney, and R. Kaplan, “Treatment options for localized prostate cancer: quality-adjusted life years and the effects of lead-time,” Urology, vol. 63, no. 1, pp. 103-109, January 2004.
[23] A. Konski, W. Speier, A. Hanlon, J. R. Beck, and A. Pollack, “Is proton beam therapy cost effective in the treatment of adenocarcinoma of the prostate?,”J. Clin. Oncol., vol. 25, no. 24, pp. 3603-8, August 2007.
[24] W. P. Smith, J. Doctor, I. J. Kalet, and M. H. Phillips, “A decision aid for intensity-modulated radiation-therapy plan selection in prostate cancer based on a prognostic Bayesian network and a Markov model,”Artif. Intell. Med., vol. 46, no. 1, pp. 119-130, 2009.
[25] E. Szumacher, H. Llewellyn-Thomas, E. Franssen, E. Chow, G. DeBoer, C. Danjoux, C. Hayter, E. Barnes, and L. Andersson, “Treatment of bone metastases with palliative radiotherapy: patients' treatment preferences.,” Int. J. Radiat. Oncol. Biol. Phys., vol. 61, no. 5, pp. 1473-81, May 2005.
[26] C. E. Pedreira, L. Macrini, M. G. Land, and E. S. Costa, “New decision support tool for treatment intensity choice in childhood acute lymphoblastic leukemia.,” IEEE Trans. Inf. Technol. Biomed., vol. 13, no. 3, pp. 284-90, May 2009.
[27] M. Morelle, E. Haslé, I. Treilleux, J.-P. Michot, T. Bachelot, F. Penault-Llorca, and M.-O. Carrère, “Cost-effectiveness analysis of strategies for HER2 testing of breast cancer patients in France.,” Int. J Technol. Assess. Health Care, vol. 22, no. 3, pp. 396-401, January 2006.
[28] D. Marshall, K. N. Simpson, C. C. Earle, and C. W. Chu, “Economic decision analysis model of screening for lung cancer.,” Eur. J. Cancer, vol. 37, no. 14, pp. 1759-67, September 2001.
[29] R. K. Khandker, J. D. Dulski, J. B. Kilpatrick, R. P. Ellis, J. B. Mitchell, and W. B. Baine, “A decision model and cost-effectiveness analysis of colorectal cancer screening and surveillance guidelines for average-risk adults.,” Int. J. Technol. Assess. Health Care, vol. 16, no. 3, pp. 799-810, January 2000.
[30] M. a J. van Gerven, F. J. Diez, B. G. Taal, and P. J. F. Lucas, “Selecting treatment strategies with dynamic limited-memory influence diagrams.,” Artif. Intell. Med., vol. 40, no. 3, pp. 171-86, July 2007.
[31] R. R. Meyer, H. H. Zhang, L. Goadrich, D. P. Nazareth, L. Shi, and W. D. D'Souza, “A multiplan treatment-planning framework: a paradigm shift for intensity-modulated radiotherapy.,” Int. J. Radiat. Oncol. Biol. Phys., vol. 68, no. 4, pp. 1178-89, July 2007.
[32] T. Hong, D. Craft, F. Carlsson, and T. Bortfeld, “Multicriteria Optimization in IMRT Treatment Planning for Locally Advanced Cancer of the Pancreatic Head,” Int J Radiat Oncol Biol Phys, vol. 72, no. 4, pp. 1208-1214, 2008.

Each of the above is incorporated herein by reference in its entirety.

TABLE 2
Summary of Cancer Treatment Selection Models in the Literature
Data Considered in Decision Models

| | | |
|---|---|---|
| Tumor Anatomy | Gleason Grade | [21], [20], [22], [24], |
| | TNM or mass | [30], [28], [24], [29] |
| | PSA | [20], [24] |
| Patients characteristics | Age | [21], [20], [22], [24], [30], [23], |
| | | [28], [26], [25] |
| | Gender | [30], [26], [25] |
| | Race | [26], [25] |
| | Treatment history | [30], [26] |
| | Comorbidities | [21] |
| | Laboratory results | [26] |
| Costs | | [30], [23], [28], [29], [25], [27] |

Decision Criteria

| | |
|---|---|
| Quality of life | [20], [22], [30], [23], [24], [25] |
| Patient Utility | [21], [22], [30], [23], [32] |
| Survival | [20], [28], [24], [29], [25] |
| Cost effectiveness | [23], [28], [29], [27] |
| Chance of metastasis | [20], [24], [27] |
| Risk of relapse | [26], [27] |
| Disutility | [20] |
| Tumor Response | [30] |
| Planning target volume (PTV) | [31], [32] |

Methods

| | |
|---|---|
| Markov framework | [21], [20], [22], [30], [23], [29] |
| Cost-Effectiveness analysis | [23], [28], [29], [27] |
| Decision trees | [28], [29], [27] |
| Bayesian Networks | [30], [24] |
| Optimization modeling | [31], [32] |
| Multivariate analysis | [25] |
| Neural Networks | [26] |

### SUMMARY

Radiation Therapy (RT) is the most commonly prescribed single agent in cancer therapeutics. Approximately, half of cancer patients receive RT as part of their treatment. There has been great improvement in the quality and effectiveness of RT delivery in the last years. Unfortunately, neoadjuvant CRT is not beneficial for all patients. The treatment response ranges from a pathologic complete response (pCR) to a resistance. It is reported that only 10 to 20 percent of patients with advanced rectal cancer show pCR to neoadjuvant CRT. Nowadays, patients with no response or minimum tumor response to neoadjuvant CRT before its initiation are not being identified.

Identifying patients that potentially could benefit from CRT and justifying a given treatment path will hopefully minimize side effects caused by the current treatment practices. We are entering in a new era of personalized, patient-specific care, and with the advent of low-cost individual genomic and proteomic analysis, we are on the path of employing patient's biologic data to systematically predict the best course of therapy.

Treatment decision making for cancer is complex. Every patient is unique with their own genetic traits, predisposition to side effects and preferences. The patient and clinician's subjective judgment plays a vital role in making sound treatment decisions. Furthermore, various patient-specific factors make it difficult to objectively and quantitatively compare various treatment decisions.

As described herein a prediction model is described that is based on the gene expression profiles of a sample of cell lines for the response of a patient to RT (Radiosensitivity) using their genomic information. Measures of the patient's individual clinical information, biological characteristics and anticipated quality of life are integrated into a patient-centered prescriptive model that determines the most appropriate course of action at a given stage (II and III) for rectal cancer.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

In one aspect, disclosed herein are methods for predicting radiation sensitivity in a subject, comprising: a) assaying a biological sample from the subject for gene expression levels of a gene panel comprising 2, 3, 4, 5, 6, 7, 8, 9, 10, or more genes selected from the group consisting of AW979276 (MAGP; SEQ ID NO: 1), Chromosome 5 Open Reading Frame 56 (C5orf56 also known as also known as IRF1 antisense RNA 1 (IRF1-AS1) (SEQ ID NO: 4)), Cystic fibrosis transmembrane conductance regulator (CFTR), Cytoplasmic FMR1 Interacting Protein 1 (CYFIP1), Hs.441600 (also known as AW592246 hf48c01.x1 Soares_NFL_T_GBC_S1 (SEQ ID NO: 2)), Hs.664912 (also known as BF515306 Hs. UI-H-BW1-ank-g-05-0-ULs1 NCI_CGAP_Sub7 (SEQ ID NO: 5), Hs.668213 (also known as BG150083 nad51d01.x1 NCI_CGAP_Lu24SEQ ID NO: 6), Interleukin-18 binding protein (IL18BP), Lysine-specific demethylase 5A (KDM5A), LOC100129195 (ZSCAN16 antisense RNA 1 (ZSCAN16-AS1)(SEQ ID NO: 3), and Ras related in brain (RAB) 13 (RAB13); b) comparing the gene expression levels to control values to generate a radiation sensitivity score; and c) treating the subject with radiation therapy when the patient has a high radiation sensitivity score and treating the subject without radiation therapy when the patient has a low radiation sensitivity score.

Also disclosed herein are kits or assays comprising primers, probes, or binding agents for detecting expression of 2, 3, 4, 5, 6, 7, 8, 9, 10, or more genes selected from the group consisting of AW979276 (MAGP)(SEQ ID NO: 1), Chromosome 5 Open Reading Frame 56 (C5orf56) (SEQ ID NO: 4)), Cystic fibrosis transmembrane conductance regulator (CFTR), Cytoplasmic FMR1 Interacting Protein 1 (CYFIP1), Hs.441600 (AW592246) (SEQ ID NO: 2), Hs.664912 (BF515306) (SEQ ID NO: 5), Hs.668213 (BG150083)(SEQ ID NO: 6), Interleukin-18 binding protein (IL18BP), Lysine-specific demethylase 5A (KDM5A), LOC100129195 (ZSCAN16-AS1)(SEQ ID NO: 3), and Ras related in brain (RAB) 13 (RAB13)

### BRIEF DESCRIPTION OF THE DRAWINGS

The components in the drawings are not necessarily to scale relative to each other. Like reference numerals designate corresponding parts throughout the several views.

FIG. 1 is a diagram of colon and rectum;

FIG. 2 is a rectal cancer detection and staging process;

FIG. 3 is the organization of this document;

FIG. 4 illustrates SF2 and transformed SF2;

FIG. 5 illustrates an example experimental design;

FIG. 6 illustrates a model performance in terms of adjusted R-square;

FIG. 7 is a decision tree prediction model;

FIG. 8 shows variable importance based on entropy reduction;

FIG. 9 is a Random Forest Algorithm;

FIG. 10 shows a Multivariate Regression Prediction Results on the Rectal Cancer dataset;

FIG. 11 shows a Random Forest Prediction Results on the Rectal Cancer dataset;

FIG. 12 shows a Multivariate Regression Prediction Results on the Esophageal Cancer dataset;

FIG. 13 shows a Random Forest Prediction Results on the Esophageal Cancer dataset;

FIG. 14A shows the characteristic function of a crisp set;

FIG. 14B the membership function of a fuzzy set;

FIG. 15 shows a degree of membership of the crisp value to the fuzzy value of the fuzzy state variable;

FIG. 16 shows Membership Functions in terms of Survival, Adverse events and Efficacy;

FIG. 17 shows a sensitivity analysis based for survival;

FIG. 18 shows a sensitivity analysis based on efficacy; and

FIG. 19 is an example operation flow chart.

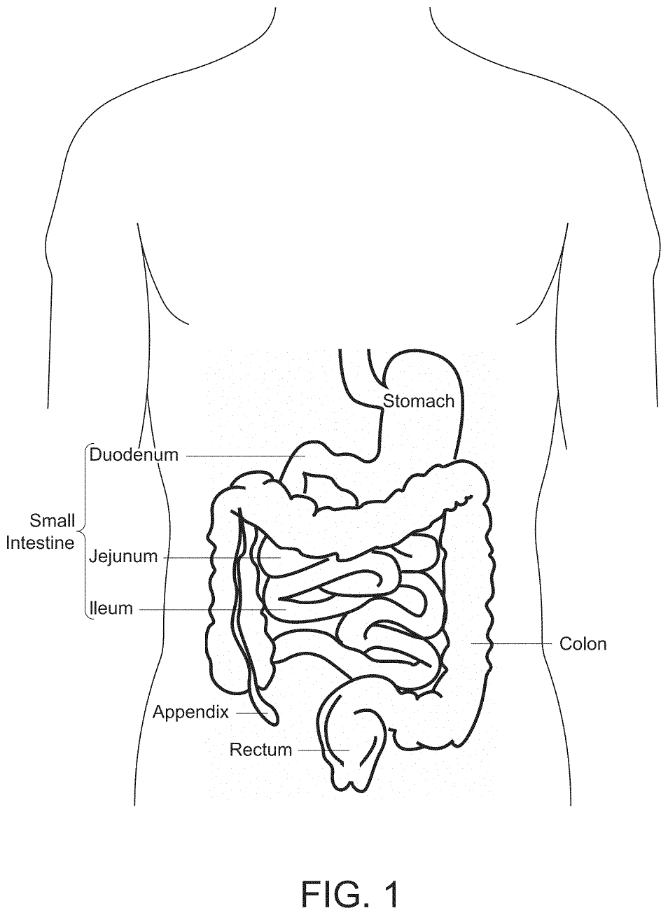

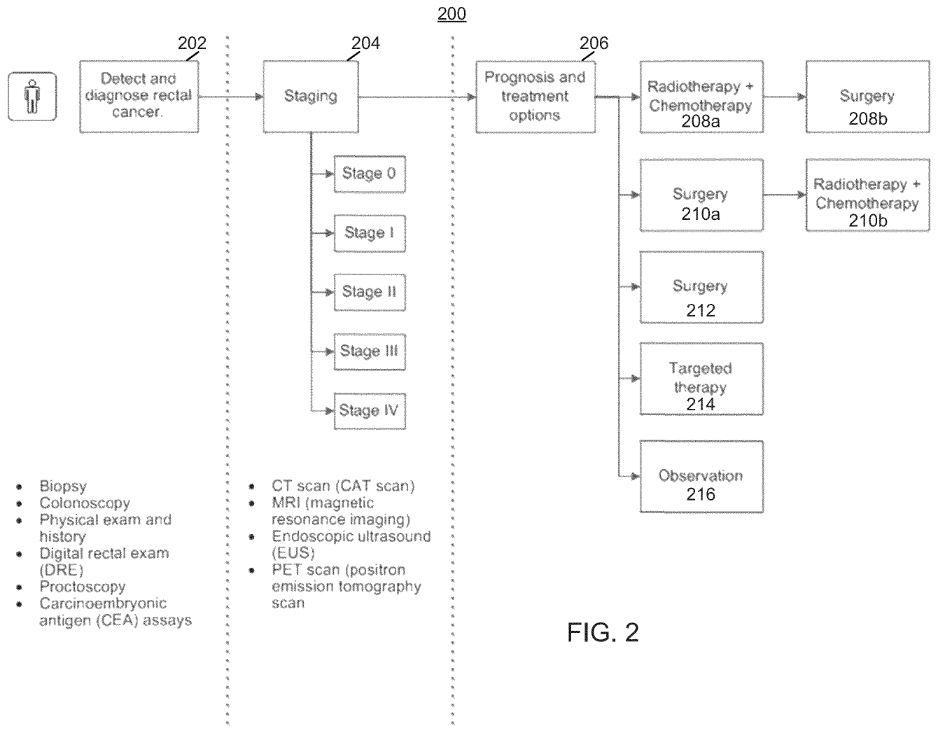

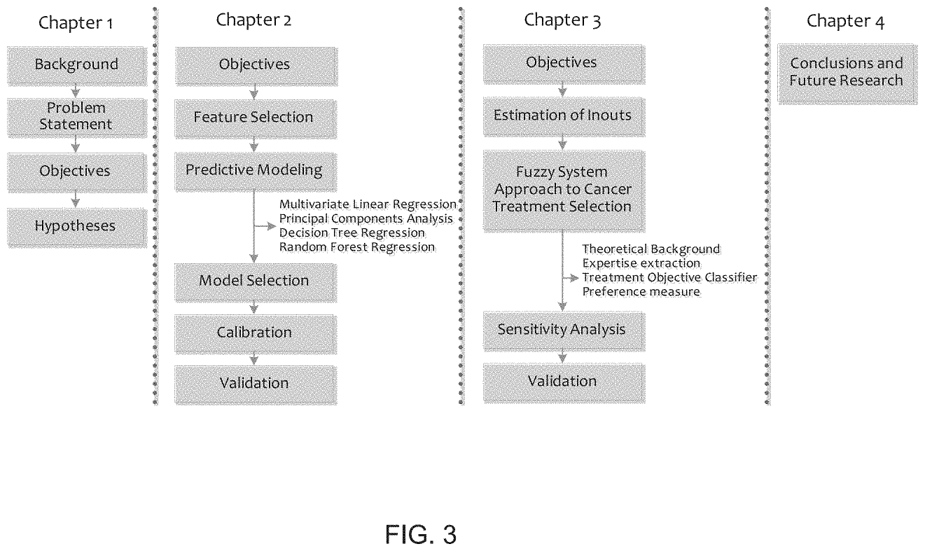

### DETAILED DESCRIPTION

Radiation therapy (RT) is the most commonly prescribed cancer treatment and can be effective in curing cancer. The success rates for RT are comparable with those achieved with surgery in some cancers (prostate, head and neck and cervical cancer). Over the past decades, RT effectiveness has improved by the discovery of physical approaches that optimizes the radiation dose to tumors and space normal tissues. With the introduction of microarrays and the use of gene expression to identify features in medical outcomes, identification of gene signatures and pathways activated in the response of cells to radiation can result in the development of treatment options which gene expression is controlled within the irradiated tumor (e.g. BUdR and IUdR were among the first classes of biological agents analyzed as radiosensitizers to enhance the effects of radiotherapy treatment).

Decision making and treatment selection in radiation oncology is subjective and based on clinic-pathological features of a large group of patient outcomes. In personalized medicine, the objective is to select the most appropriate course of treatment that fits an individual patient's needs and characteristics. Genomic medicine technological advancements has now the potential of predicting a patient predisposition to RT. Microarrays technology is one of the most widely adopted methods of genomics analyses. Microarrays experiments generate functional data on a genome-wide scale, and can provide important data for biological interpretation of genes and their functions.

The complexity and dimensionality of the data generated from gene expression microarray technology requires advanced computational approaches. Machine learning and supervised learning methods provide tools to develop predictive models from available data, and it is effective when dealing with large amounts of biological data. In this dissertation, we present a methodology to organize and analyze gene expression data and test whether it results in an accurate predictive model of tumor radiosensitivity.

Machine learning refers to the type of computational techniques that are used to develop a “model” from a set of observations of a system. The term “model” assumes that there exists an approximate relationships between the parameters considered in the system. The goal is to predict a quantitative (regression) or qualitative (classification) outcome using a set of attributes or features. Consequently, supervised learning refers to the subset of machine learning methods where the input-output relationship is assumed to be known.

Supervised learning is commonly used in the computational biology area ranging from gene expression data to analysis of interactions between biological subjects. Some of the most commonly used supervised learning methods used in computational biology include: neural networks, support vector machine, logistic regression, multivariate linear regression, decision tree-based models and ensembles (random forest). A review of these methods is presented in the following section.

Below is a discussion on the development of a personalized diagnostic tool to predict radiotherapy (RT) efficacy using the patient genomic information and estimate likelihood of response to RT of an individual patient. Later, the results of this model will be implemented into a decision model with the objective of guiding the patient and physician decision on the selection of a cancer treatment strategy.

### Review of Prediction Models in Computational Biology

A summary of the methods, relevant literature, strengths, limitations and opportunities are presented in Table 3. Artificial neural networks (ANN) and support vector machines are among the most commonly used black box machine learning tools in the literature. ANN-based approaches may be applied for classification, predictive modelling and biomarker identification within data sets of high complexity.

Below is a key to the references noted in Table 3:
[40] L. J. Lancashire, D. G. Powe, J. S. Reis-Filho, E. Rakha, C. Lemetre, B. Weigelt, T. M. Abdel-Fatah, a R. Green, R. Mukta, R. Blamey, E. C. Paish, R. C. Rees, I. O. Ellis, and G. R. Ball, “A validated gene expression profile for detecting clinical outcome in breast cancer using artificial neural networks.,” Breast Cancer Res. Treat., vol. 120, no. 1, pp. 83-93, February 2010.
[41] G. Sateesh Babu and S. Suresh, “Parkinson's disease prediction using gene expression—A projection based learning meta-cognitive neural classifier approach,” Expert Syst. Appl., vol. 40, no. 5, pp. 1519-1529, April 2013.
[42] H.-L. Chou, C.-T. Yao, S.-L. Su, C.-Y. Lee, K.-Y. Hu, H.-J. Terng, Y.-W. Shih, Y.-T. Chang, Y.-F. Lu, C.-W. Chang, M. L. Wahlqvist, T. Wetter, and C.-M. Chu, “Gene expression profiling of breast cancer survivability by pooled cDNA microarray analysis using logistic regression, artificial neural networks and decision trees.,” BMC Bioinformatics, vol. 14, no. 1, p. 100, March 2013.
[43] A.-M. Lahesmaa-Korpinen, Computational approaches in high-throughput proteomics data analysis, no. 169.2012, pp. 3-18.
[44] M. P. Brown, W. N. Grundy, D. Lin, N. Cristianini, C. W. Sugnet, T. S. Furey, M. Ares, and D. Haussler, “Knowledge-based analysis of microarray gene expression data by using support vector machines.,” Proc. Natl. Acad. Sci. U S. A., vol. 97, no. 1, pp. 262-7, January 2000.
[45] A. Statnikov, C. F. Aliferis, I. Tsamardinos, D. Hardin, and S. Levy, “A comprehensive evaluation of multicategory classification methods for microarray gene expression cancer diagnosis.,” Bioinformatics, vol. 21, no. 5, pp. 631-43, March 2005.
[46] J. Khan, J. S. Wei, M. Ringnér, L. H. Saal, M. Ladanyi, F. Westermann, F. Berthold, M. Schwab, C. R. Antonescu, C. Peterson, and P. S. Meltzer, “Classification and diagnostic prediction of cancers using gene expression profiling and artificial neural networks.,” Nat. Med., vol. 7, no. 6, pp. 673-9, June 2001.
[47] N. R. Pal, K. Aguan, A. Sharma, and S. Amari, “Discovering biomarkers from gene expression data for predicting cancer subgroups using neural networks and relational fuzzy clustering.,” BMC Bioinformatics, vol. 8, p. 5, January 2007.
[48] M. C. O'Neill and L. Song, “Neural network analysis of lymphoma microarray data: prognosis and diagnosis near-perfect.,” BMC Bioinformatics, vol. 4, p. 13, April 2003.
[49] J. S. Wei, B. T. Greer, F. Westermann, S. M. Steinberg, C. Son, Q. Chen, C. C. Whiteford, S. Bilke, A. L. Krasnoselsky, N. Cenacchi, D. Catchpoole, F. Berthold, M. Schwab, and J. Khan, “Prediction of clinical outcome using gene expression profiling and artificial neural networks for patients with neuroblastoma.,” Cancer Res., vol. 64, no. 19, pp. 6883-91, October 2004.
[50] a. Narayanan, E. C. Keedwell, J. Gamalielsson, and S. Tatineni, “Single-layer artificial neural networks for gene expression analysis,” Neurocomputing, vol. 61, pp. 217-240, October 2004.
[51] A. Ben-Hur, C. S. Ong, S. Sonnenburg, B. Scholkopf, and G. Ratsch, “Support vector machines and kernels for computational biology.,”PLoS Comput. Biol., vol. 4, no. 10, p. e1000173, October 2008.
[52] K.-B. Duan, J. C. Rajapakse, H. Wang, and F. Azuaje, “Multiple SVM-RFE for gene selection in cancer classification with expression data.,” IEEE Trans. Nanobioscience, vol. 4, no. 3, pp. 228-34, September 2005.
[53] V. Bevilacqua, P. Pannarale, M. Abbrescia, C. Cava, A. Paradiso, and S. Tommasi, “Comparison of data-merging methods with SVM attribute selection and classification in breast cancer gene expression.,” BMC Bioinformatics, vol. 13 Suppl 7, no. Suppl 7, p. S9, January 2012.
[54] L. Chen, J. Xuan, R. B. Riggins, R. Clarke, and Y. Wang, “Identifying cancer biomarkers by network-constrained support vector machines.,” BMC Syst. Biol., vol. 5, no. 1, p. 161, January 2011.
[55] M. Hassan and R. Kotagiri, “A new approach to enhance the performance of decision tree for classifying gene expression data.,” BMC Proc., vol. 7, no. Suppl 7, p. S3, December 2013.
[56] G. Dong and Q. Han, “Mining Accurate Shared Decision Trees from Microarray Gene Expression Data for Different Cancers.”
[57] G. R. Varadhachary, Y. Spector, J. L. Abbruzzese, S. Rosenwald, H. Wang, R. Aharonov, H. R. Carlson, D. Cohen, S. Karanth, J. Macinskas, R. Lenzi, A. Chajut, T. B. Edmonston, and M. N. Raber, “Prospective gene signature study using microRNA to identify the tissue of origin in patients with carcinoma of unknown primary.,” Clin. Cancer Res., vol. 17, no. 12, pp. 4063-70, June 2011.
[58] L. Schietgat, C. Vens, J. Struyf, H. Blockeel, D. Kocev, and S. Dzeroski, “Predicting gene function using hierarchical multi-label decision tree ensembles.,” BMC Bioinformatics, vol. 11, p. 2, January 2010.
[59] M. E. Ross, X. Zhou, G. Song, S. A. Shurtleff, K. Girtman, W. K. Williams, H. Liu, R. Mahfouz, S. C. Raimondi, N. Lenny, A. Patel, and J. R. Downing, “Classification of pediatric acute lymphoblastic leukemia by gene expression profiling,” Blood, vol. 102, no. 8, pp. 2951-2959, 2003.
[60] S. Salzberg, A. L. Delcher, H. Fasman, and J. Henderson, “A Decision Tree System for Finding Genes in DNA,” J. Comput. Biol., vol. 5, no. 4, pp. 667-80, 1998.
[61] C. R. Williams-DeVane, D. M. Reif, E. C. Hubal, P. R. Bushel, E. E. Hudgens, J. E. Gallagher, and S. W. Edwards, “Decision tree-based method for integrating gene expression, demographic, and clinical data to determine disease endotypes.,”BMC Syst. Biol., vol. 7, no. 1, p. 119, January 2013.
[62] J. S. Barnholtz-Sloan, X. Guan, C. Zeigler-Johnson, N. J. Meropol, and T. R. Rebbeck, “Decision tree-based modeling of androgen pathway genes and prostate cancer risk.,” Cancer Epidemiol. Biomarkers Prev., vol. 20, no. 6, pp. 1146-55, June 2011.
[63] D. Che, Q. Liu, K. Rasheed, and X. Tao, Software Tools and Algorithms for Biological Systems, vol. 696. New York, N.Y.: Springer New York, 2011, pp. 191-199.
[64] G. Stiglic, S. Kocbek, I. Pernek, and P. Kokol, “Comprehensive decision tree models in bioinformatics.,”PLoS One, vol. 7, no. 3, p. e33812, January 2012.
[65] G. J. Mann, G. M. Pupo, A. E. Campain, C. D. Carter, S.-J. Schramm, S. Pianova, S. K. Gerega, C. De Silva, K. Lai, J. S. Wilmott, M. Synnott, P. Hersey, R. F. Kefford, J. F. Thompson, Y. H. Yang, and R. a Scolyer, “BRAF mutation, NRAS mutation, and the absence of an immune-related expressed gene profile predict poor outcome in patients with stage III melanoma.,”J. Invest. Dermatol., vol. 133, no. 2, pp. 509-17, February 2013.
[66] A. Natarajan, G. G. Yardimci, N. C. Sheffield, G. E. Crawford, and U. Ohler, “Predicting cell-type-specific gene expression from regions of open chromatin.,” Genome Res., vol. 22, no. 9, pp. 1711-22, September 2012.
[67] S. C. Smith, A. S. Baras, D. Ph, G. Dancik, Y. Ru, K. Ding, C. A. Moskaluk, J. Lehmann, M. Stöckle, A. Hartmann, and K. Jae, “molecular nodal staging of bladder cancer,” vol. 12, no. 2, pp. 137-143, 2013.
[68] A. Schaefer, M. Jung, H.-J. Mollenkopf, I. Wagner, C. Stephan, F. Jentzmik, K. Miller, M. Lein, G. Kristiansen, and K. Jung, “Diagnostic and prognostic implications of microRNA profiling in prostate carcinoma.,” Int. J. Cancer, vol. 126, no. 5, pp. 1166-76, March 2010.
[69] J. Zhu, “Classification of gene microarrays by penalized logistic regression,” Biostatistics, vol. 5, no. 3, pp. 427-443, July 2004.
[70] S. K. Shevade and S. S. Keerthi, “A simple and efficient algorithm for gene selection using sparse logistic regression,” Bioinformatics, vol. 19, no. 17, pp. 2246-2253, November 2003.
[71] M. J. Hassett, S. M. Silver, M. E. Hughes, D. W. Blayney, S. B. Edge, J. G. Herman, C. a Hudis, P. K. Marcom, J. E. Pettinga, D. Share, R. Theriault, Y.-N. Wong, J. L. Vandergrift, J. C. Niland, and J. C. Weeks, “Adoption of gene expression profile testing and association with use of chemotherapy among women with breast cancer.,” I Clin. Oncol., vol. 30, no. 18, pp. 2218-26, June 2012.
[72] M. a Cobleigh, B. Tabesh, P. Bitterman, J. Baker, M. Cronin, M.-L. Liu, R. Borchik, J.-M. Mosquera, M. G. Walker, and S. Shak, “Tumor gene expression and prognosis in breast cancer patients with 10 or more positive lymph nodes.,” Clin. Cancer Res., vol. 11, no. 24 Pt 1, pp. 8623-31, December 2005.
[73] a L. Richards, L. Jones, V. Moskvina, G. Kirov, P. V Gejman, D. F. Levinson, a R. Sanders, S. Purcell, P. M. Visscher, N. Craddock, M. J. Owen, P. Holmans, and M. C. O'Donovan, “Schizophrenia susceptibility alleles are enriched for alleles that affect gene expression in adult human brain.,” Mol. Psychiatry, vol. 17, no. 2, pp. 193-201, February 2012.
[74] C. C.-M. Chen, H. Schwender, J. Keith, R. Nunkesser, K. Mengersen, and P. Macrossan, “Methods for identifying SNP interactions: a review on variations of Logic Regression, Random Forest and Bayesian logistic regression.,” IEEE/ACM Trans. Comput. Biol. Bioinform., vol. 8, no. 6, pp. 1580-91, 2011.
[75] E. B. Hunt, Concept learning, an information processing problem. New York: Wiley, 1962.
[76] L. Breiman, J. Friedman, C. Stone, and R. Olshen, Classification and Regression Trees. California: Wadsworth International, 1984.
[77] L. Breiman, “Random Forest,” Mach. Learn., vol. 45, pp. 5-32, 2001.
[78] P. Geurts, A. Irrthum, and L. Wehenkel, “Supervised learning with decision tree-based methods in computational and systems biology.,”Mol. Biosyst., vol. 5, no. 12, pp. 1593-605, December 2009.

Each of the above is incorporated herein by reference in its entirety.

More recent studies using ANN approaches in system biology include: a validated a reduced (from 70 to 9 genes) gene signature capable of accurately predicting distant metastases by Lancashire et al [40]; a model to predict Parkison's disease using micro-array gene expression data by Sateesh Babu et al [41]; and a gene expression-based model to select 20 genes that are closely related to breast cancer recurrence by Chou et al [42].

The support vector machine (SVM) algorithm consists on a hyperplane or a set of hyperplanes in a high-dimensional space, which are then used for classification or regression [43]. Support vector machines (SVM) have a number of mathematical features that make them attractive for gene expression analysis due to its ability of dealing with large data sets with high data dimensionality, ability to identify outliers, flexibility in choosing a similarity function and sparseness of the solution [44]. According to Statnikov et al, multi-category SVM are the most effective classifiers in performing accurate cancer diagnosis using gene expression data [45]. Most studies conclude that the main limitation of SVM is the lack of interpretability of the results and heuristic determination of the Kernel parameters.

TABLE 3
Summary of prediction models in computational biology

| | Relevant | | Limitations (L) |
|---|---|---|---|
| Method | Literature | Advantages | Opportunities (O) |
| Artificial | [40]-[42], | Can process data | (L)Hard to interpret |
| neural | [46]-[50] | containing non- | (O) Sensitivity analysis and rule |
| networks | | linear | extraction can be used extract knowledge |
| | | relationships and | (L) Prone to over-fitting |
| | | interactions | (O) re-sampling and cross-validation can |
| | | Can handle noisy | be used to address this issue |
| | | or incomplete | (L) Multiple solutions associated with |
| | | data | local minima |
| | | Capable of | |
| | | feature selection | |
| | | in high | |
| | | dimensional data | |
| | | Good predictive | |
| | | performance | |
| Support | [44], [45], | Can process data | (L) Large margin classifiers are known to |
| vector | [51]-[54] | containing non- | be sensitive to the way features are scaled |
| machines | | linear | (O) data normalization |
| and kernels | | relationships and | (L) sensitive to unbalanced data |
| | | interactions | (O) assign a different misclassification |
| | | Can provide a | cost to each class |
| | | good out-of- | (L) Kernel parameters are data-dependent |
| | | sample | (O) Try a linear and a non-linear kernel |
| | | generalization | (L) Prone to over-fitting |
| | | Optimality | (O) Local alignment kernel |
| | | problem is | |
| | | convex | |
| Decision | [55]-[64] | Readily | (L) Classification performance of a single |
| tree-based | | understandable | tree lower than other methods |
| methods | | Interpretable | (O1) Classification performance could be |
| and | | Ability to rank | improved by combining more than two |
| Random | | the attributes | features at each node |
| forest | | according to their | (O2) Classification performance is |
| | | relevance in | improved by aggregation of predictions |
| | | predicting the | by ensembles |
| | | output | (L) Decision trees are sensitive to the |
| | | | training data set used and overfitting |
| | | | (O) Random forest use bootstrapping to |
| | | | estimate outcomes by aggregation of |
| | | | difference trees |
| | | | (L) Inadequate to perform regression of |
| | | | continuous values |
| | | | (O) Tree ensembles use a large number |
| | | | of tree to obtained aggregated solutions |
| | | | and good performance |
| Logistic | [65]-[74] | Most commonly | (L) LR can only be used to predict |
| regression | | used method in | discrete functions |
| | | classifications | (L) Parameter estimation procedure of |
| | | problems | LR assumes an adequate number of |
| | | Often used as | samples for each combination of |
| | | benchmark to | independent variables |
| | | compare models | (O) Needs to make sure a large sample |
| | | Can handle | size and determine adequate number of |
| | | nonlinear effect, | samples for each combination |
| | | interaction effect | (L) Independent binary variable must be |
| | | and power terms | balanced |
| | | Readily | (O) Resample the available data to obtain |
| | | understandable | a balanced dataset |
| | | Interpretable | |

In models using logistic regression for classification, the outcome of interest is assumed to be binomially distributed with the logistic function f(y)=1/(1+exp^{-y}). The variable y is a measure of the contributions of the parameters y=β₀+β₁x₁+ . . . +βₙxₙ where β₀ is a constant term and the β₁, β₂, . . . , βₙ are regression coefficients. Models [65]-[74] include [paragraph still in process]

The origin of tree-based learning methods is often credited to Hunt [75], but the method became recognized in the field of statistics by Breiman et al. [76] with the Classification And Regression Trees (CART). Since then, more decision-tree based methods have been proposed to improve the prediction accuracy by aggregating the predictions given by several decision trees for the same outcome. Although decision tree models were originally designed to address classification problems, they have been extended to handle Univariate and multivariate regression. Random forests (RF) models [77] is a randomization method that modifies the node splitting of the CART procedure as follows: at each node, K candidate variables are selected at random among all input candidate variables, an optimal candidate test is found for each of these variables, and the best test among them is eventually selected to split the node [78].

Below is a comparison of supervised learning methods appropriate to the structure and objectives of the models. Based on the performance of the models, a prediction model trained in tumor cell gene expression data is validated in two independent clinical outcomes datasets for patients that received pre-operative RT.

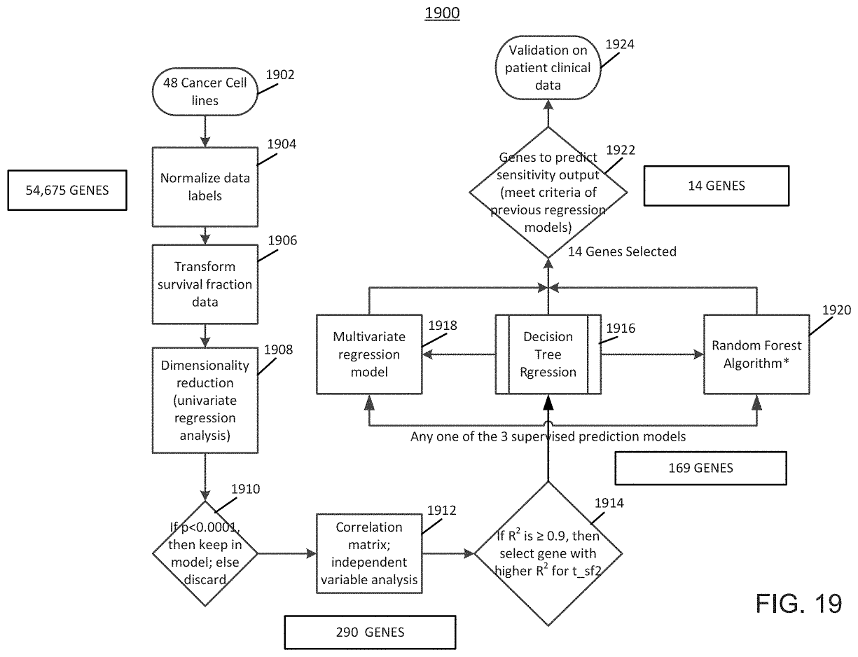

With referenced to FIG. 19, there is shown an operational flow 1900 to predict radiation sensitivity (Radiosensitivity), defined based on cellular clonogenic survival after 2 Gy (SF2) for 48 cell lines (1902, see

Table 4). Since gene expression profiles are available for all cell lines, gene expression is used as the basis of the prediction model. The operational flow 1900 may be predicated on two hypotheses. The first is that a radiosensitivity cell-based prediction model can be validated using clinical patient data from rectal and esophagus cancer patients that received RT before surgery. The second is that a radiosensitivity genomic-based prediction model could identify patients with rectal cancer that may benefit from RT treatment by assigning higher values of SF2 to radio-resistant patients and lower values of SF2 to radio-sensitive patients.

As evidence, radiosensitivity is defined based on cellular clonogenic survival after 2 Gy (SF2) for 48 cell lines (1902). Since gene expression profiles are available for all cell lines, gene expression is used as the basis of the prediction model. Radiosensitivity prediction has been studied, and a clinically validated radiosensitivity index (RSI) has been defined to estimate radiosensitivity. The approach herein differs from conventional methods in that the response SF2 transformation process and the gene expression selection process use a statistically based procedure versus a biological feature selection approach.

### Methods and Materials

Sample: Cell lines are used to construct the prediction model and were obtained from the NCI [35]. Cells were cultured as recommended by the NCI in Roswell Park Memorial Institute medium (RPMI) 1640 supplemented with glutamine (2 mmol/L), antibiotics (penicillin/streptomycin, 10 units/mL) and heat-inactivated fetal bovine serum (10%) at 37° C. with an atmosphere of 5% CO2.

Microarrays: analyses using microarrays technology has been widely adopted for generating gene expression data on a genomic scale. Gene expression profiles were from obtained from Affymetrix U133plus chips from a previously published study by S. Eschrich, H. Zhang, H. Zhao, D. Boulware, J.-H. Lee, G. Bloom, and J. F. Torres-Roca, “Systems biology modeling of the radiation sensitivity network: a biomarker discovery platform.,” Int. J. Radiat. Oncol. Biol. Phys., vol. 75, no. 2, pp. 497-505, October 2009.

Output: The survival fraction at 2 Gy (SF2) of 48 human cancer cell lines used in the classifier was obtained from Torres-Roca, 2005 and are presented in Table 4.

The procedure used to obtain these values consisted on cells being plated so that 50 to 100 colonies would form per plate and incubated overnight at 37° C. to allow for adherence. Cells were then radiated with 2 Gy using a Cesium Irradiator. Exposure time was adjusted for decay every 3 months. After irradiation, cells were incubated for 10 to 14 days at 37° C. before being stained with crystal violet. Only colonies with at least 50 cells were counted. The values for SF2 were determined using the following equation 1:

$$SF 2 = \frac{number of colonies}{total number of cells plated \times plating efficiency} \qquad ( 1 )$$

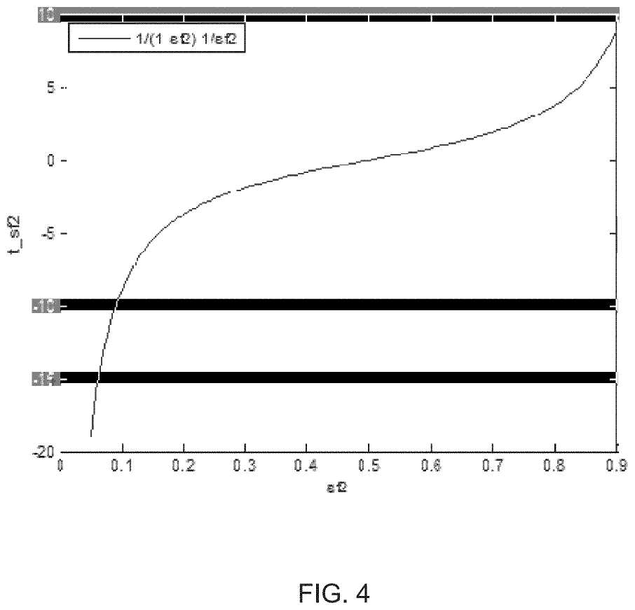

Output transformation: A transformation function (equation 2) is applied to the SF2. Originally SF ranges between 0 and 1; with the transformation functions, SF2 can range between −∞ and ∞. The objective of this transformation is to enhance the extremes values of SF2 (radio-sensitive and radio-resistant responses). The transformation follows equation 2 and is represented in FIG. 4, which illustrates SF2 and transformed SF2

TABLE 4
SF2 measured values for 48 cell lines (1902) in the database

| | Tissue of | Measured | | Tissue of | Measured |
|---|---|---|---|---|---|
| Cell Line | Origin | SF2 | Cell Line | Origin | SF2 |
| Breast_bt549 | Breast Cancer | 0.632 | Leuk_ccrfcem | Leukemia | 0.185 |
| Breast_hs578t | Breast Cancer | 0.79 | Leuk_hl60 | Leukemia | 0.315 |
| Breast_mcf7 | Breast Cancer | 0.576 | Leuk_molt4 | Leukemia | 0.05 |
| Breast_mdamb231 | Breast Cancer | 0.82 | Melan_loximvi | Melanoma | 0.68 |
| Breast_t47d | Breast Cancer | 0.52 | Melan_m14 | Melanoma | 0.42 |
| Breast_mdamb435 | Breast Cancer | 0.1795 | Melan_malme3m | Melanoma | 0.8 |
| Cns_sf268 | Central Nervous | 0.45 | Melan_skmel2 | Melanoma | 0.66 |
| | System Cancer | | | | |
| Cns_sf539 | Central Nervous | 0.82 | Melan_skmel28 | Melanoma | 0.74 |
| | System Cancer | | | | |
| Cns_snb19 | Central Nervous | 0.43 | Melan_skmel5 | Melanoma | 0.72 |
| | System Cancer | | | | |
| Cns_snb75 | Central Nervous | 0.55 | Melan_uacc257 | Melanoma | 0.48 |
| | System Cancer | | | | |
| Cns_u251 | Central Nervous | 0.57 | Melan_uacc62 | Melanoma | 0.52 |
| | System Cancer | | | | |
| Colon_colo205 | Colon Cancer | 0.69 | Ovar_skov3 | Ovarian Cancer | 0.9 |
| Colon_hcc-2998 | Colon Cancer | 0.44 | Ovar_ovcar4 | Ovarian Cancer | 0.29 |
| Colon_hct116 | Colon Cancer | 0.38 | Ovar_ovcar5 | Ovarian Cancer | 0.408 |
| Colon_hct15 | Colon Cancer | 0.4 | Ovar_ovcar8 | Ovarian Cancer | 0.6 |
| Colon_ht29 | Colon Cancer | 0.79 | Ovar_ovcar3 | Ovarian Cancer | 0.55 |
| Colon_km12 | Colon Cancer | 0.42 | Prostate_du145 | Prostate Cancer | 0.52 |
| Colon_sw620 | Colon Cancer | 0.62 | Prostate_pc3 | Prostate Cancer | 0.484 |
| Nsclc_a549atcc | Non-Small Cell | 0.61 | Renal_7860 | Renal Cancer | 0.66 |
| | Lung Cancer | | | | |
| Nsclc_ekvx | Non-Small Cell | 0.7 | Renal_a498 | Renal Cancer | 0.61 |
| | Lung Cancer | | | | |
| Nsclc_hop62 | Non-Small Cell | 0.164 | Renal_achn | Renal Cancer | 0.72 |
| | Lung Cancer | | | | |
| Nsclc_hop92 | Non-Small Cell | 0.43 | Renal_caki1 | Renal Cancer | 0.37 |
| | Lung Cancer | | | | |
| Nsclc_ncih23 | Non-Small Cell | 0.086 | Renal_sn12c | Renal Cancer | 0.62 |
| | Lung Cancer | | | | |
| Nsclc_h460 | Non-Small Cell | 0.84 | Renal_uo31 | Renal Cancer | 0.62 |
| | Lung Cancer | | | | |

### Feature Selection

Standard prediction models and variable reduction methods face an important challenge with the dimensionality of the data. This is the case for the area of genomic applications where the number of genes is considerably higher than the samples available to study them. In this problem, a total of m=54,675 potential candidates (gene expression) are considered to be part of the prediction models with a total of n=48 observations tumor cells. The most commonly used approaches, such as PCA, require for n>m. However, this problem shows m>>n. Thus, a methodology to reduce the sample size and to identify features that are statistically independent (low correlation values) is recommended. The objectives of the dimension reduction procedure presented here are to:
Identify independent (not highly correlated) features
Improve performance of prediction models by removing irrelevant predictors
Improve efficiency of modeling using fewer features
Reduce the selection of effects whose influence on dependent variable is mostly random

The approach herein is a Univariate method that selects the most relevant (statistically significant) features one by one and excluding the rest. This technique is computationally simple and fast to process high-dimensional datasets, and it is independent of the classification/regression models. When using this procedure, feature dependencies are ignored. Thus, a step to extract independent features has to be included (step 5 below).

Thus, with reference to FIG. 19, the procedure to select the candidate predictors includes:

Start: 54,675 gene expressions

| | |
|---|---|
| 1. | Merge repeated gene expression by replacing with average |
| 2. | Normalize labels in datasets to create a single data file (1904 - |
| | Cell-lines have different labels in the various files) |
| 3. | Conduct response variable transformation (1906) |
| | ${T}_{S F 2} = \frac{1}{1 - S F 2} - \frac{1}{S F 2} ( 2 )$ |
| 4. | Perform univariate regression with each gene versus T_SF2 (1908): |
| | If (p-value >= 0.0001) then Variable is kept in the model; |
| | Otherwise, variable is excluded (1910) |
| 5. | Identify independent variable |
| | i. Estimate correlation matrix (1912) |
| | ii. If (correlation coefficient >= 0.9) then select gene with higher |
| | R² for t_sf2 in cluster (1914); |
| | iii. Otherwise, consider this variable “independent”. |

End: The reduced data set contained 169 features (gene expressions).

The dimension reduction process presented in this study is also compared with two other feature selection methods including random forests and support vector machines. Since the subset of selected features is different for all methods there is no evidence to support one method over the other.

### Predictive Model Development

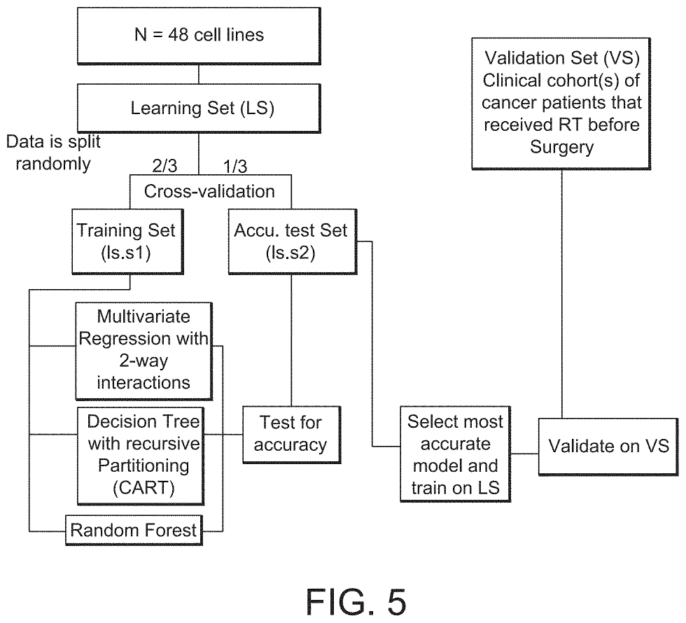

Predictive models are developed and compared based on their performance. The experimental design of the models is presented in Figure. The process to build, test and validate the models has been used in the literature of supervised learning methods in computational and systems biology, and it can be summarized as follows:
Learning sample (LS) consists of 48 cell lines
Build model on LS using the default parameterization of the method using cross-validated: 2/3 learning sample (ls.s1), 1/3 testing sample (ls.s2)
Evaluate the accuracy of model on the test sample ls.s2
If the accuracy results are not acceptable, then play with different values of the parameter K (for random forest)
   Select the value K* that leads to best accuracy on S2.
Build selected model on LS and validate predictions on TS to get an estimate Acc_{final} of its accuracy. There are two TS datasets and will be described in the validation section. FIG. 5 illustrates an experimental design.

In the selection of a prediction model after 1914, there is tradeoff between simplicity and wholeness. Simpler models can be more understandable, computationally tractable. On the other hand, more complex models tend to fit the data better and to capture more information from available data. Two simple models (a Multivariate regression model and a decision tree model) and a more complex model (random forest) are created and compared to select the most appropriate model in the prediction of radiation sensitivity.

Model 1: Multivariate Regression with 2-Way Interactions (1918)

Linear regression is a method used in building models from data for which dependencies can be closely approximated and predicting the value of a response (y) from a set of predictors (xᵢ). Let x₁, x₂, . . . , x₁₆₉ be a set of 169 predictors believed to be associated with the transformed response T_SF2. The linear regression model for the j^{th} has the form given by (3):

T_SF2ⱼ=β₀+β₁xⱼ₁+β₂xⱼ₂+ . . . +β₁₆₉x₁₆₉+∈ⱼ (3)

The matrix notation is ŷ=Xβ. Where E is a random error with E(∈ⱼ)=0, Var(∈ⱼ)=σ², Cov(∈ⱼ,∈ₖ)=0 ∀j≠k, and βᵢ, i=0, 1, . . . , 169 are the regression coefficients. The approach to estimate the vector β's in this study is the least square estimation: The value of β that minimizes the sum of square residuals (Y−Xβ)′(Y−Zβ) and the decomposition is given by (4):

$${\sum }_{j = 1}^{n} {( {y}_{j} - \overline{y} )}^{2} = {\sum }_{j} {( \hat{y} - \overline{y} )}^{2} + {\sum }_{j} {\hat{ϵ}}^{2} \qquad ( 4 )$$

The goodness of fit (GOF) of the model is measured by the proportion of the variability that the model can explain given by R². The formulation and motivation of the use of R² and other performance measures of GOR have been extensively addressed in the literature [84].

The creation of the multivariate regression model allowed for 2-way interactions to be considered as predictors in the regression model. The steps to build the models are as follows: (1) The model was coded using proc glmselect in SAS 9.3. (2) The selection process consisted on a stepwise forward selection (effects already in the model do not necessarily stay as the fit is iteratively tested considering all candidate variables). The decision criteria used considers the optimal value of the Akaike information criterion (AIC) and the adjusted R² to access the trade-off between the GOF of the model and the number of predictors in the system. The AIC value is given by AIC=2k−2 ln(L), where k is the number of parameters and L is the value of the likelihood function.

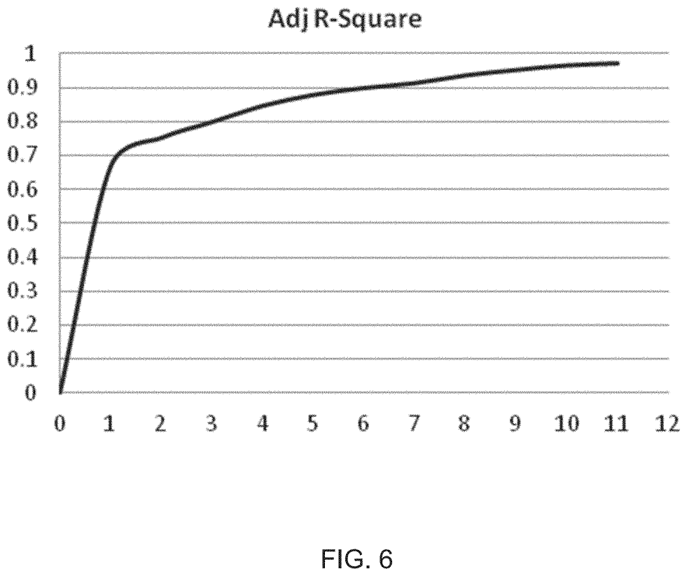

The value of the adjusted R² is also presented in Thus, FIG. 6. It can be observed that the value for the adjusted R² does not considerably improve after step 7; therefore the total number of interaction effects in the model is eight. A summary of the selection process and significant predictors' interactions, parameter estimates and performance measures (AIC and adjusted R²) can be found in Table 5.

TABLE 5
Multivariate regression model selection

| | Interaction of effects | | Parameter | Number of effects | adjusted | |
|---|---|---|---|---|---|---|
| Step | (gene expression) | | estimate | in model | R² | AIC |
| 0 | intercept | 1 | 58.207248 | 1 | 0 | 184.8924 |
| 1 | 222868_s [symbol] | 1554636_a [symbol] | −1.976624 | 2 | 0.6657 | 133.5468 |
| 2 | 226367_a [symbol] | 244039_x_ [symbol] | −1.916222 | 3 | 0.7498 | 120.9651 |
| 3 | 208923_a [symbol] | 1557248_a [symbol] | −0.187086 | 4 | 0.7967 | 112.4197 |
| 4 | 243559_a [symbol] | 1564276_a [symbol] | 1.555853 | 5 | 0.8443 | 101.1404 |
| 5 | 236687_a [symbol] | 1564128_a [symbol] | −2.664955 | 6 | 0.8766 | 91.5949 |
| 6 | 215703_a [symbol] | 1557062_a [symbol] | 0.833148 | 7 | 0.897 | 84.6667 |
| 7 | 202252_a [symbol] | 238735_at | −0.132294 | 8 | 0.9112 | 79.3727* |

[symbol]

indicates data missing or illegible when filed

Thus, FIG. 6 illustrates a model performance in terms of adjusted R-square.

### Model 2: Decision Tree (1916)

A decision tree induction is a method of data analysis that maps the dependency relationships in the data, and it is sometimes subsumed by the category of cluster analyses. The goal with CART is to build a regression tree and predict radiosensitivity (SF2) based on the gene expression profiles available using recursive partitioning or rpart in R. The following steps are followed to build the tree in rpart:

1. Splitting criteria: is given that the split of a node A into two sons A_{R} and A_{L} is (5):

P(A_{L})r(A_{L})+P(A_{R})r(A_{R})≤P(A)r(A) (5)

Where: P(A) is the probability of A for future observations, and r(A) is the risk of A. However, rpart considers measures of impurity or diversity for the note splitting criteria. Let f be the impurity function defined by (6):

$$I ( A ) = {\sum }_{i = 1}^{C} f ( {p}_{iA} ) \qquad ( 6 )$$

Where p_{iA} is the proportion of the elements in A that belong to class i. Therefore, if I(A)=0 when A is pure, f must be concave with f(0)=f(1)=0. the split with the maximal impurity reduction (the Gini or information index) is used.

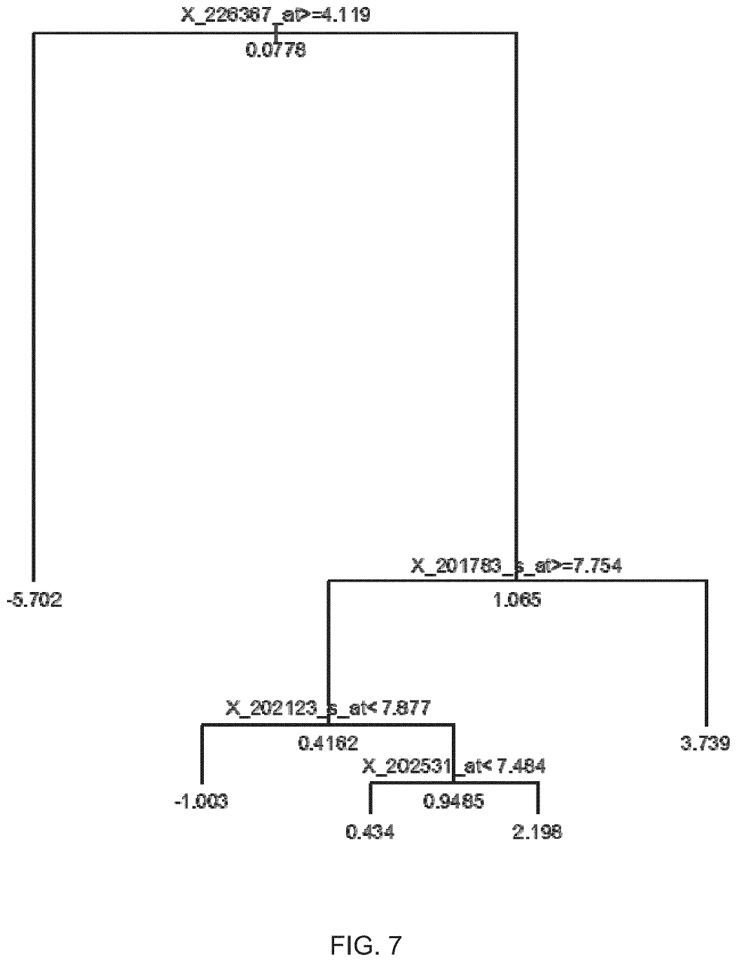

FIG. 7 illustrates and example decision tree prediction model in accordance with the present disclosure.

### Model 3: Random Forest (1920)

Supervised learning provides techniques to learn predictive models only from observations of a system and is therefore well suited to deal with the highly experimental nature of biological knowledge.

Breiman's Random Forests algorithm [77] builds each tree from a bootstrap sample like Bagging but modifies the node splitting procedure as follows: at each test node, K attributes are selected at random among all input attributes, an optimal candidate test is found for each of these attributes, and the best test among them is eventually selected to split the node.

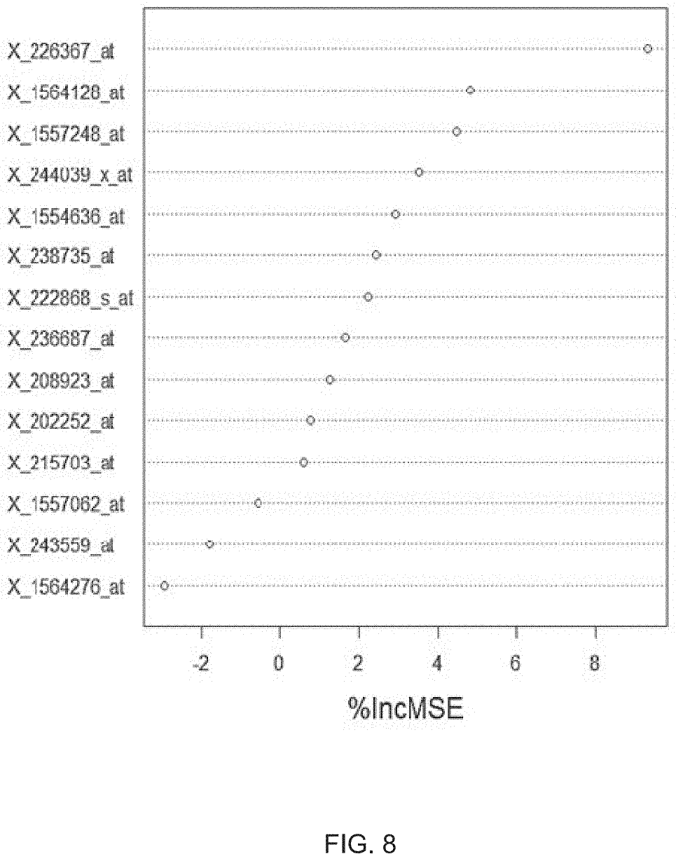

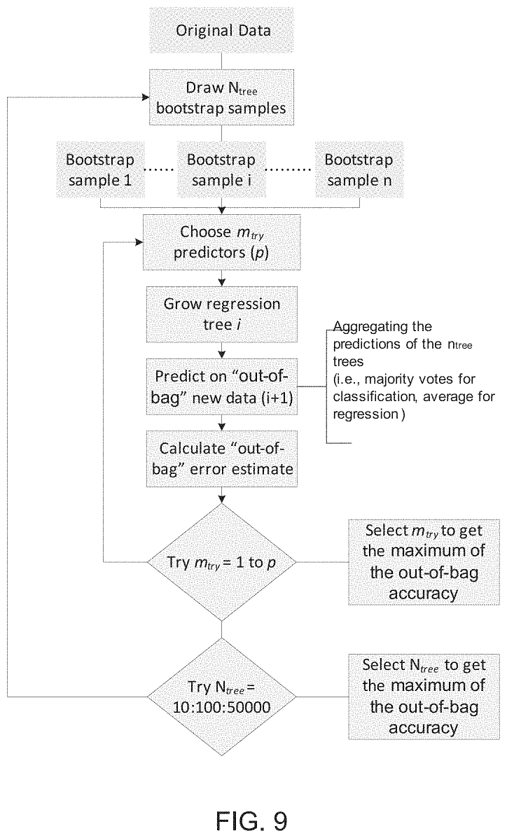

The prediction model for radiosensitivity was built using the random forest package in R (1922). The selected predictors (gene expression profiles), ranked in the order the variable reduced prediction error, are presented FIG. 8, which shows variable importance based on entropy reduction. The algorithm used to build the prediction model is a Random Forest Algorithm, as shown in FIG. 9.

### Validation (1924)

The predictive models were validated in three independent datasets. Clinical Outcomes are classified into responder (R) and non-responder (NR).

Rectal Cancer Dataset
Sample size: 20 patients.
Test of ETA1=ETA2 vs ETA1 not=ETA2 is significant at 0.0185 using the random forest model and 0.003144 using regression model (See).

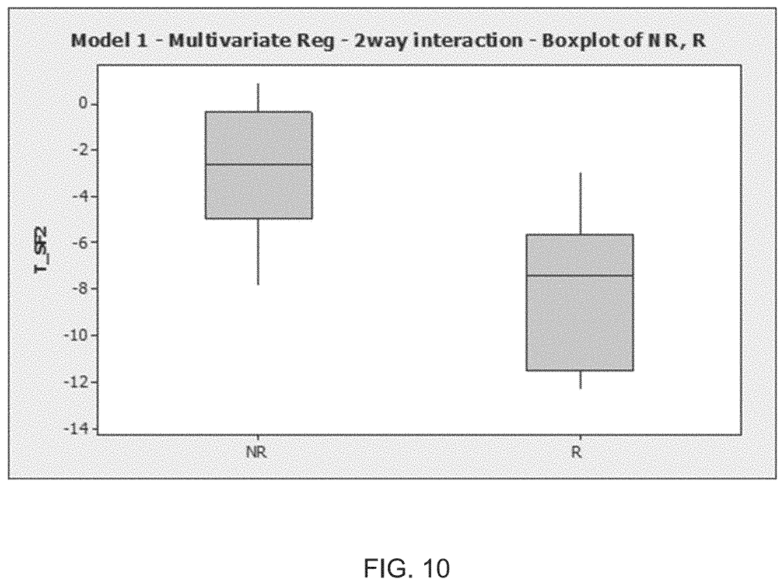

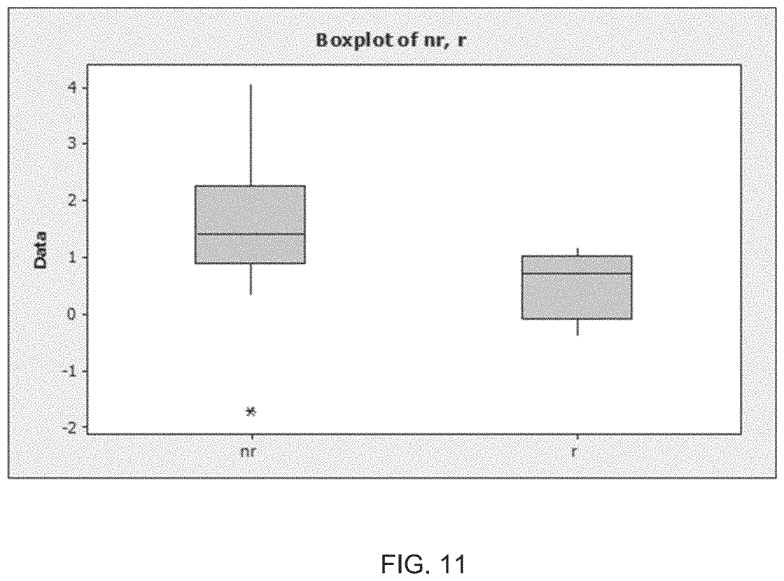

FIG. 10 shows a Multivariate Regression Prediction Results on the Rectal Cancer dataset. FIG. 11 shows a Random Forest Prediction Results on the Rectal Cancer dataset.

Esophageal Cancer Dataset
Sample size: 12 patients.
Test of ETA1=ETA2 vs ETA1 not=ETA2 is significant at 0.047 using the random and 0.0032 using regression model (See).

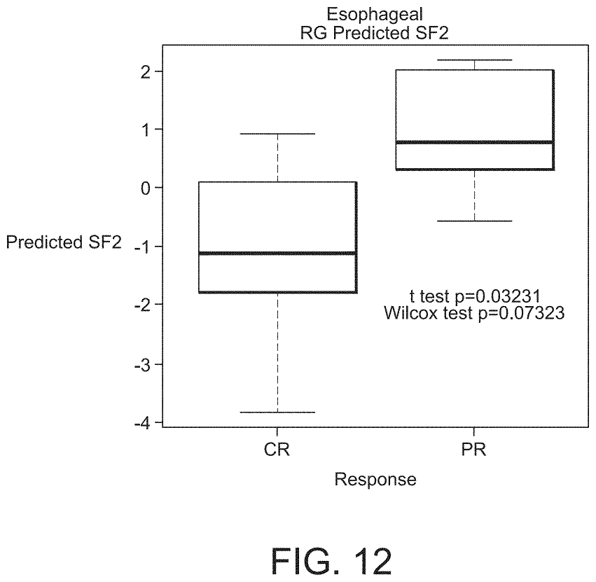

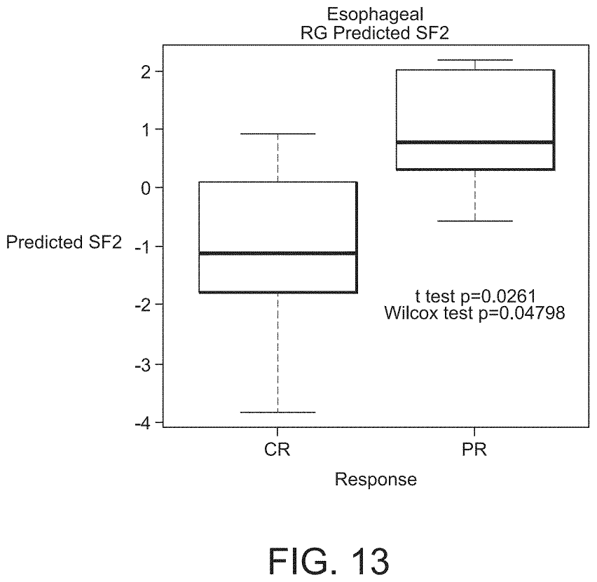

FIG. 12 shows a Multivariate Regression Prediction Results on the Esophageal Cancer dataset. FIG. 13 shows a Random Forest Prediction Results on the Esophageal Cancer dataset.

### Discussion

Herein, the microarray gene expression data processing and prediction model is built following four steps:

(1) Response variable transformation: SF2 for 48 cancer cell lines was transformed using a mathematical function to augment the lower and upper extremes (related to Radiosensitive and Radioresistant cell lines) of the radiosensitivity/radioresistance spectrum

(2) Dimensionality reduction: candidate gene expression probesets were selected using a univariate regression analysis with statistical significance (p<=0.001)

(3) Model building: Breiman's Random Forest algorithm [77] which is an ensemble of decision trees, was trained using the learning sample of the 48 human cancer cell lines to predict the transformed SF2

(4) Model calibration: statistically significant differences (p<0.05) were found between the median of the training set of the cell lines and the validation set of patients. We estimated the calibration parameters based on the calculated difference in medians.

Thus, the above provides clinical support for a practical and novel assay to predict tumor radiosensitivity. Due to the difference in experimental measurement in DNA microarray gene expression values among different cohorts, calibration methods may be created to standardize validation across different sites. Further testing of this technology in larger clinical populations is also supported.

### A Fuzzy Approach for Treatment Selection in Cancer Treatment

An implementation of the above is a model based design and decision making of a multiple-input/multiple-output (MIMO) fuzzy logic controller (FLC). FLC defines a static nonlinear control law by employing a set of fuzzy if-then rules (also known as fuzzy rules). A set of fuzzy rules is derived via knowledge acquisition and reflects the knowledge of an expert in the area where the decision making is made. Below is an introduction to basic FLC related concepts involving the definitions of a fuzzy sets, fuzzy input, fuzzy output variables and fuzzy state space. Next, the types of FLCs are presented which include the Takagi-Sugeno, Mamdani and the sliding mode FLC models. Finally, the decision model is presented to select the most appropriate treatment based on the individual characteristics of the patient.

Classical sets are refer to as crisp sets in fuzzy set theory to differentiate them from fuzzy sets. A crisp set C of the universe of discourse, or domain D, can be represented by using its characteristic function

The function μ_{C}:D→[0,1] is a characteristic function of the set C if and only if for all d

$${μ}_{C} ( d ) = \{ \begin{matrix}1 if d ∈ C \\ 0 if d ∉ C\end{matrix}$$

Therefore, for crisp sets every element of d of D either d∈C, or d∉C. It is not the same for fuzzy sets. Given a fuzzy set F, it is not necessary that d∈F, or d∉F. This function can be generalized to a membership function which assigns every d∈C a value from the unit interval [0,1] instead from the two element set {0,1}.

The membership function μ_{F} of a fuzzy set F is a function defined as μ_{F}: D→[0,1]. Every element d∈D has a membership degree μ_{F}(d)∈[0,1]. Thus, the fuzzy set F is completely determined by:

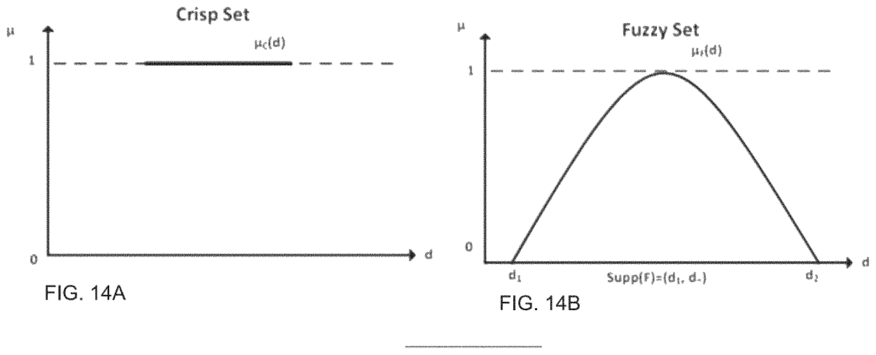

F={(d,μ_{F}(d))|d∈D}

Where D and F are continuous domains, and μ_{F} is a continuous membership function. FIGS. 14A and 14B show the characteristic function of a crisp set and the membership function of a fuzzy set respectively. Support of F denoted as supp(F) refers to the elements of D that have degrees of membership to F.

Herein, only fuzzy sets with convex membership functions are considered. A fuzzy set F is convex if and only if:

Vx,y∈XVλ∈[0,1]: μ_{A}(λ·x+(1−λ)·y)≥min(μ_{A}(x),μ_{A}(y))

The FLC described here have uses inputs and output variables whose states variables are x₁, x₂, . . . , xₙ. Let X be a given closed interval of reals, a state variable xᵢ with values in the fuzzy sets are fuzzy state variables, and the set of these fuzzy values are called term-set. The values xᵢ are denoted as TXᵢ, and the j-th value of the i-th fuzzy state is denoted as LXᵢⱼ. Each LXᵢⱼ defined by a membership function:

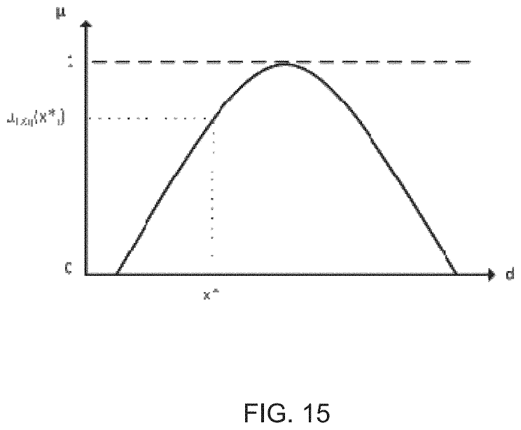

LXᵢⱼ=∫_{X}μ_{X}(x)/x

Where μ_{X}(x)/x is the degree of membership of the crisp value xᵢ* of xᵢ to the fuzzy value LXᵢⱼ of xᵢ. FIG. 15 shows a degree of membership of the crisp value to the fuzzy value of the fuzzy state variable

The fuzzy values LXᵢⱼ₋₁ and LXᵢⱼ₊₁ are referred to as the left and right neighbor of the fuzzy value LXᵢⱼ respectively. Also, It is required that each fuzzy value shares a certain degree of membership with its left and right neighbor:

supp(LXᵢⱼ₋₁)∩supp(LXᵢⱼ)≠Ø

supp(LXᵢⱼ)∩supp(LXᵢⱼ₊₁)≠Ø

μ_{LX}_{_{ij−1}}(x)+μ_{LX}_{ᵢⱼ}(x)=1

μ_{LX}_{ᵢⱼ}(x)+μ_{LX}_{ᵢⱼ₊₁}(x)=1

Given a fuzzy state vector x=(x₁, x₂, . . . , xₙ)^{T}, each xᵢ takes some fuzzy value LXᵢ∈TXᵢ. Therefore, a random fuzzy state vector can be written as LX=(LX₁, LX₂, . . . , LXₙ)^{T}. Each fuzzy state variable takes its fuzzy values amongst the elements of a finite term-set; therefore, there is a finite number of different fuzzy state vectors, denoted as LXⁱ (for I=1, 2, . . . , M). The center of a fuzzy region, LXⁱ=(LX₁ⁱ, LX₂ⁱ, . . . , LXₙⁱ)^{T} defined by the crisp state vector xⁱ=(x₁ⁱ, x₂ⁱ, . . . , xₙⁱ)^{T} ∈Xⁿ, where xₖⁱ are crisp values such that μ_{LX}_{ᵢⱼ}(x₁ⁱ)=1, μ_{LX}_{ᵢⱼ}(x₂ⁱ)=1, . . . , μ_{LX}_{ᵢⱼ}(xₙⁱ)=1.

The general form of a model is given as \dot{x}=f(x, u), where f is a n×1 state vector and u is the n×1 input vecto, and let u=g(x) be the control law. Then, we can estimate the closed loop system as \dot{x}=f(x, g(x)).

Bayesian Decision Theory/models are appropriate for groups of patients but are complicated in application to individual patient factors. Fuzzy set theory effectively handles the deterministic uncertainty and subjective information of clinical decision making. Other decision-making approaches include neural networks, utility theory, statistical pattern matching, decision trees, rule-based systems, and model-based schemes. Fuzzy set theory has been successfully used alone or combined with neural networks and expert systems to solve challenging biomedical problems in practice
Fuzzy Logic
Probabilistic methods for uncertain reasoning
Classifiers and statistical learning methods
Neural networks
Control theory
Languages
Current Cancer Treatment Selection Process

Thus, in view of the above, the present disclosure seeks to develop an expert decision knowledge-based system that is able to effectively depict patient preferences and evaluate rectal cancer treatment options. The present disclosure further seeks to integrate patient-centered measures into a decision model that considers multiple criteria. This may be based on the following, non-limiting hypotheses:
decision procedures implemented in the model can use language and mechanisms suitable for human interpretation and understanding
The physician and the patient can jointly use these models to compare different medical interventions and make a decision on choosing the appropriate intervention for the patient.
The decision model is capable of providing a decision by weighting conflictive objectives for the treatment outcomes.
The decision framework allows decision makers to modify priorities for the various criteria/objectives considered to make the selection of treatments.

### Fuzzy Discrete Event System Approach

A focus herein may be the selection of three cancer treatment regimens for stage II and stage III rectal cancer patients that will receive treatment for the first time (no metastasis):
Surgery alone
Radiation and Surgery (either neoadjuvant and adjuvant)
No treatment

There are 27 possible combinations (3×3×3=27), 9 transition matrices for the 3 regimens. Semi-Gaussian functions are used to produce gradual changes of membership/probability (see Table 6). The essential elements of an effective cancer treatment regimen include:
Selecting a treatment sufficiently intense increase chances of survival and reduces rate of recurrence
Minimizing treatment toxicity and adverse effects
Selecting a treatment that a patient that can cure or eliminate the cancer tumor

TABLE 6
Decision model elements and membership functions

| Decision Criteria | Category | Membership Function |
|---|---|---|
| 5 yr. Survival rate | High | $\{ \begin{matrix}1 , x > 85 \\ {e}^{- \frac{1}{2} {( \frac{x - 85}{5} )}^{2}} , x \leq 85\end{matrix}$ |
| | Medium | ${e}^{- \frac{1}{2} {( \frac{x - 55}{6} )}^{2}} , - ∞ < x < ∞$ |
| | Low | $\{ \begin{matrix}{e}^{- \frac{1}{2} {( \frac{x - 55}{5} )}^{2}} , x > 55 \\ 1 , x \leq 55\end{matrix}$ |
| Adverse events | 3^{rd} grade | $\{ \begin{matrix}1 , x > 45 \\ {e}^{- \frac{1}{2} {( \frac{x - 45}{5} )}^{2}} , x \leq 45\end{matrix}$ |
| | 2^{nd} grade | ${e}^{- \frac{1}{2} {( \frac{x - 30}{6} )}^{2}} , - ∞ < x < ∞$ |
| | 1^{st} grade | $\{ \begin{matrix}{e}^{- \frac{1}{2} {( \frac{x - 20}{5} )}^{2}} , x > 20 \\ 1 , x \leq 20\end{matrix}$ |
| Efficacy | Likely | $\{ \begin{matrix}1 , x > 85 \\ {e}^{- \frac{1}{2} {( \frac{x - 85}{5} )}^{2}} , x \leq 85\end{matrix}$ |
| | Neutral | ${e}^{- \frac{1}{2} {( \frac{x - 65}{6} )}^{2}} , - ∞ < x < ∞$ |
| | unlikely | $\{ \begin{matrix}{e}^{- \frac{1}{2} {( \frac{x - 45}{5} )}^{2}} , x > 45 \\ 1 , x \leq 45\end{matrix}$ |

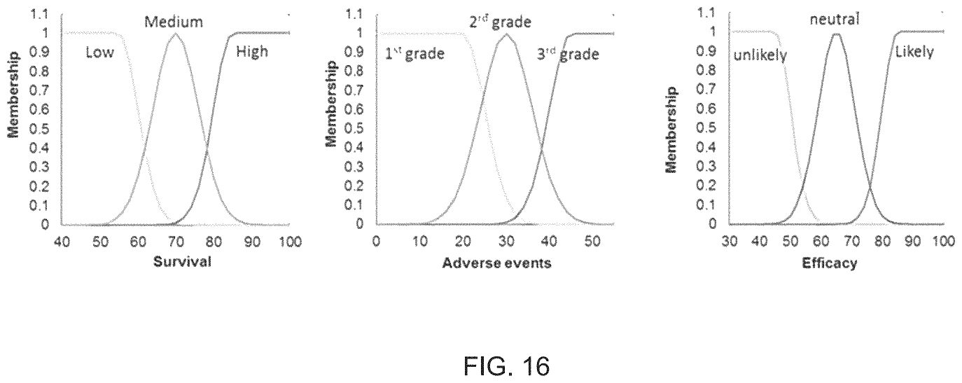

FIG. 16 shows Membership Functions in terms of Survival, Adverse events and Efficacy. The decision function, E(h), is defined as the weighted average of the new state vectors:

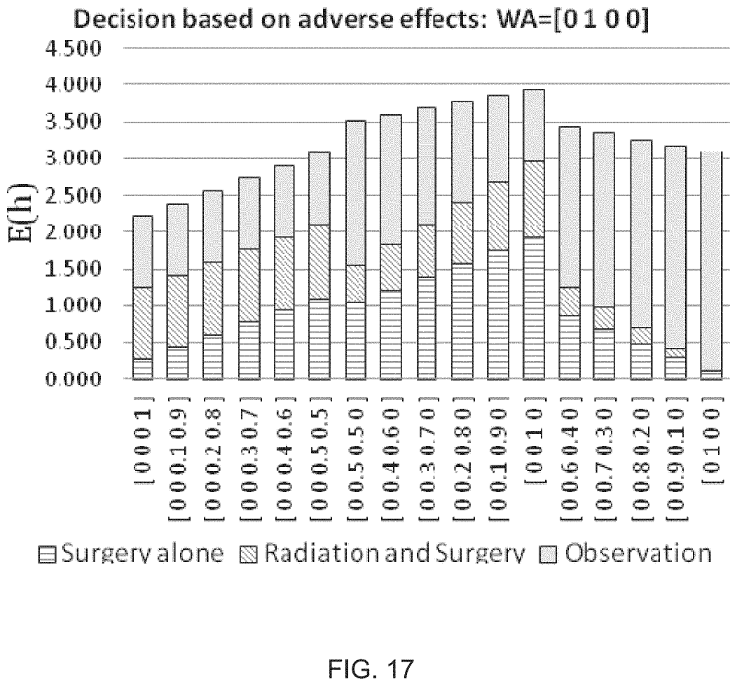

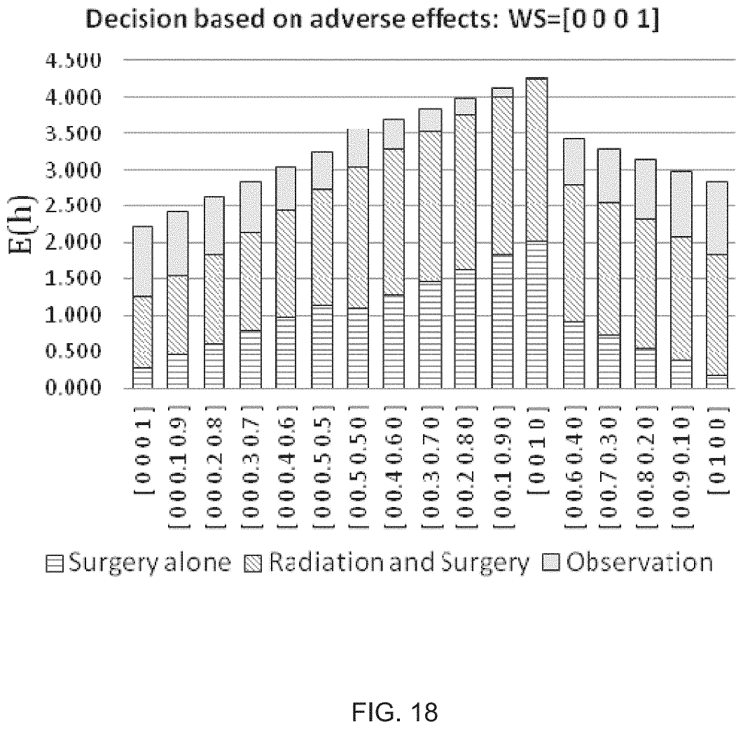

E(h)=α·W_{S}+β·W_{A}·γ·W_{E} (3)

where W_{S}, W_{A} and W_{E} are the weight vectors for survival, adverse effects and treatment efficacy. FIG. 17 shows a sensitivity analysis based for survival based on the above. FIG. 18 shows a sensitivity analysis based on efficacy based on the above.

### Conclusion

In accordance with the methods above, the mathematical model to predict radio sensitivity is able to discriminate team responders and nonresponders using expression data for 14 genes, as listed below. In addition, a subset of these 14 genes as also able to predict radiotherapy sensitivity with statistical significance. It is noted that the number of genes in the model is selected based on model performance, and the best model as achieved with the 14 genes below.

The List of the 14 Genes are:

| Probe set | Gene symbol |
|---|---|
| 238735_at | AW979276 also known as MAGP Homo sapiens |
| | mRNA sequence (SEQ ID NO: 1) |
| 1564276_at | C5orf56 also known as IRF1 antisense RNA 1 (IRF1- |
| | AS1)(SEQ DI NO: 4) |
| 215703_at | CFTR |
| 208923_at | CYFIP1 |
| 244039_x_at | Hs.441600 also known as AW592246 or hf48c01.x1 |
| | Soares_NFL_T_GBC_S1 (SEQ ID NO: 2) |
| 243559_at | Hs.664912 also known as BF515306 Hs. UI-H-BW1- |
| | ank-g-05-0-UI.s1 NCI_CGAP_Sub7 (SEQ ID NO: 5) |
| 236687_at | Hs.668213 also known as BG150083 nad51d01.x1 |
| | NCI_CGAP_Lu24 (SEQ ID NO: 6) |
| 222868_s_at | IL18BP |
| 226367_at | KDM5A |
| 1557062_at | LOC100129195 also known as ZSCAN16 antisense |
| | RNA 1 (ZSCAN16-AS1) (SEQ ID NO: 3) |
| 202252_at | RAB13 |
| 1554636_at | Gene symbol name not available |
| 1557248_at | Gene symbol name not available |
| 1564128_at | Gene symbol name not available |

For the random forest, the 14 genes are used to run the prediction since several (random) trees with different subset of genes are grown in order to get an aggregate prediction. However, we can rank the variables that are the best predictors (as they reduce the prediction error)(see FIG. 8).

For the regression model, one can see in the every step of the modeling and how the performance changes as new variables are added to the model. A model may be built that only considers the first 5 steps.

TABLE 7
Multivariate Regression

| | Interaction of effects | | Parameter | Number of effects | Adj. | |
|---|---|---|---|---|---|---|
| Step | (gene expression) | | estimate | in model | R² | AIC |
| 0 | intercept | 1 | 58.21 | 1 | 0 | 184.89 |
| 1 | 222868_s_at | 1554636_at | −1.97 | 2 | 0.6657 | 133.54 |
| 2 | 226367_at | 244039_x_at | −1.92 | 3 | 0.7498 | 120.96 |
| 3 | 208923_at | 1557248_at | −0.18 | 4 | 0.7967 | 112.41 |
| 4 | 243559_at | 1564276_at | 1.55 | 5 | 0.8443 | 101.14 |
| 5 | 236687_at | 1564128_at | −2.66 | 6 | 0.8766 | 91.59 |
| 6 | 215703_at | 1557062_at | 0.83 | 7 | 0.897 | 84.66 |
| 7 | 202252_at | 238735_at | −0.13 | 8 | 0.9112 | 79.37* |

The 14 genes or output after running the multivariate regression (see, FIG. 19): Model selection using stepwise forward selection. Given a set of candidate models for the data, the preferred model is the one with the minimum AIC value and adjusted R-square (not the highest one but when the improvement is not significant when adding more variables (or genes)).

Models are built on data from 48 cell lines of different tumors (breast, colon, etc.). Once a final model is selected, we tested on patients that received Radiation, and based on the gene expression of the tumor, we tested how our model is able to discriminate between responders and non-responders.

It should be understood that the various techniques described herein may be implemented in connection with hardware or software or, where appropriate, with a combination of both. Thus, the methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computer, the machine becomes an apparatus for practicing the presently disclosed subject matter. In the case of program code execution on programmable computers, the device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A method for predicting radiation sensitivity in a subject, comprising:
a) assaying a biological sample from the subject for gene expression levels of a gene panel comprising 2, 3, 4, 5, 6, 7, 8, 9, 10, or more genes selected from the group consisting of AW979276 (MAGP)(SEQ ID NO: 1), Chromosome 5 Open Reading Frame 56 (C5orf56) (SEQ ID NO: 4)), Cystic fibrosis transmembrane conductance regulator (CFTR), Cytoplasmic FMR1 Interacting Protein 1 (CYFIP1), Hs.441600 (AW592246) (SEQ ID NO: 2), Hs.664912 (BF515306) (SEQ ID NO: 5), Hs.668213 (BG150083)(SEQ ID NO: 6), Interleukin-18 binding protein (IL18BP), Lysine-specific demethylase 5A (KDM5A), LOC100129195 (ZSCAN16-AS1)(SEQ ID NO: 3), and Ras related in brain (RAB) 13 (RAB13);
b) comparing the gene expression levels to control values to generate a radiation sensitivity score; and
c) treating the subject with radiation therapy when the patient has a high radiation sensitivity score and treating the subject without radiation therapy when the patient has a low radiation sensitivity score.

2. The method of claim 1, wherein the biological sample is assayed using a microarray comprising two or more oligonucleotide probe sets selected from the group consisting of 238735_at, 1564276_at, 215703_at, 208923_at, 244039_x_at, 243559_at, 236687_at, 222868 s at, 226367_at, 1557062_at, and 202252_at.

3. The method of claim 1, wherein the biological sample is further assayed for gene expression levels of one or more genes detectable by oligonucleotide probe sets selected from the group consisting of 1554636_at, 1557248_at, and 1564128_at.

4. The method of claim 1, wherein the gene expression levels are analyzed by multivariate regression analysis or principal component analysis to calculate the risk score.

5. A kit or assay comprising primers, probes, or binding agents for detecting expression of 6, 7, 8, 9, 10, or more genes selected from the group consisting of AW979276, C5orf56, CFTR, CYFIP1, Hs.441600, Hs.664912, Hs.668213, IL18BP, KDM5A, LOC100129195, and RAB13.

6. The kit of claim 5, comprising two or more oligonucleotide probe sets selected from the group consisting of 238735_at, 1564276_at, 215703_at, 208923_at, 244039_x_at, 243559_at, 236687_at, 222868 s at, 226367_at, 1557062_at, and 202252_at.

7. The kit of claim 5, further comprising two or more oligonucleotide probe sets selected from the group consisting of 1554636_at, 1557248_at, and 1564128_at.

8. A method to predict radiation sensitivity, comprising:
identifying a predetermined number of cancer cell lines;
normalizing labels in datasets associated with the predetermined number of cancer cell lines to create a single data file;
conducting a response variable transformation function to the signal data file;
performing a univariate regression with each gene versus a survival fraction (T_SF2), wherein if a p-value is greater than or equal to a predetermined value, a variable is kept in the model;
identifying an independent variable;
estimating a correlation matrix wherein if a correlation coefficient is greater than or equal to a second predetermined value, a gene is selected with a higher R² for t_SF2; and
applying a supervised prediction model to the gene.

9. The method of claim 8, wherein
$$SF 2 = \frac{number of colonies}{total number of cells plated \times plating efficiency}$$

10. The method of claim 8, wherein the response variable transformation function is defined as: T_SF2=1/(1−SF2)−1/SF2.

11. The method of claim 8, wherein the predetermined value is 0.0001.

12. The method of claim 8, wherein the second predetermined value is 0.9.

13. The method of claim 8, the applying a supervised prediction model to the gene further comprising applying one of a Multivariate regression, Decision tree or Random forest model.

14. The method of claim 13, wherein 2, 3, 4, 5, 6, 7, 8, 9, 10, or more genes are selected from the group consisting of AW979276, C5orf56, CFTR, CYFIP1, Hs.441600, Hs.664912, Hs.668213, IL18BP, KDM5A, LOC100129195, and RAB13.
